(19) United States
(12) Patent Application Publication
(10) Pub. No.: US 2026/0079154 A1
(43) Pub. Date: Mar. 19, 2026

(54) COMPOSITION, DEVICE, SYSTEM, AND METHODS FOR DETECTING ENVIRONMENTAL TOXICANTS

(71) Applicant: Arizona Board of Regents on Behalf of the University of Arizona, Tucson, AZ (US)

(72) Inventors: Jeong-Yeol Yoon, Tucson, AZ (US); Lane Breshears, Tucson, AZ (US); Kelly Reynolds, Tucson, AZ (US)

(21) Appl. No.: 19/110,006

(22) PCT Filed: Sep. 12, 2023
(86) PCT No.: PCT/US2023/073990

§ 371 Date: Mar. 7, 2025

Related U.S. Application Data
(60) Provisional application No. 63/405,778, filed on Sep. 12, 2022.

(51) Int. Cl. G01N 33/53; G01N 33/52

(52) U.S. Cl. CPC G01N 33/5308; G01N 33/526; G01N 2430/60

(57) ABSTRACT

Disclosed herein are aspects of a composition for detecting environmental toxicants such as, but not limited to, perfluorinated-alkyl substance, perfluorooctanoic acid, and the like. The composition allows for the rapid, sensitive detection of perfluorinated-alkyl substances (PFAS) by utilizing the competitive interactions during capillary action. The composition comprising one or more cellulose fibers; and one or more biomolecules associated with the one or more cellulose fibers, wherein the composition is configured so that when the composition contacts the liquid sample, the one or more biomolecules disassociate from the one or more cellulose fibers by interacting with the one or more environmental toxicants thereby decreasing the flow of the liquid sample comprising one or more environmental toxicants. A system for using the composition is also disclosed along with a method of using the paper microfluidic chips and a method making the paper microfluidic chip.

### CROSS REFERENCE TO RELATED APPLICATION

This application claims the benefit of, and priority to, U.S. Provisional Application No. 63/405,778, filed Sep. 12, 2022, which is incorporated herein by reference in its entirety.

### FIELD

The disclosure concerns a device for detecting environmental toxicants, and a system comprising the device, and a method of using the device.

### BACKGROUND

Per- and polyfluoroalkyl substances (PFASs) are receiving prominent attention due to their highly inert properties and potential danger to human health. PFASs are a large group of thousands of chemicals characterized by a carbon chain that is either fully fluorinated (perfluoro-) or partially fluorinated (polyfluoro-). These C-F polar covalent bonds are one of the strongest chemical bonds in organic chemistry. This makes a PFAS a compound that does not degrade rapidly, if at all, and instead remains in the environment over time. Accordingly, PFASs are often referred to as “forever chemicals.” Over the thousands of existing PFASs, each receives its specific identity and properties based on its functional group or some substitutions in its carbon chain.

As early as 1950, PFASs became the go-to chemical used in many products, industries, and processes. They can repel water, oil, and soil, provide chemical and thermal stability, and/or reduce friction. However, because of their very proficient and widespread use and longevity in the environment, they have become an environmental problem and a concerning health issue.

Typical testing methods for PFASs require collecting a sample on-site and sent to a laboratory capable of performing HPLC-MS/MS. This method is extremely sensitive and will find the exact molecular structure of any components present in the sample. The method can detect various PFAS chemicals in amounts as low as 1 ag/μL. However, there are several issues with this method including its complexity and cost. It requires not only expensive machinery but also extensive training to perform the procedure and analyze the results. Another limitation to this method is the necessity of returning samples to laboratories for testing. This method additionally requires a specific column coated with reagents to isolate the particular PFAS molecules. As previously mentioned, PFASs is a vast family of compounds. Therefore, much research must be conducted to expand the existing detection methods for accurately estimating this diverse chemical group.

Other than HPLC-MS/MS, methylene blue (cationic dye) has also been used to detect anionic surfactant PFOA. The simple nature of this reaction can lead to rapid and low-cost assay. However, its limit of detection (LOD) is 10 pg/μL (10 ppb)—much higher than the EPA advisory level of 70 ppt. It is also not specific since methylene blue can bind to other anionic surfactants. Finally, it cannot detect cationic, zwitterionic, or non-ionic PFAS molecules.

A molecularly imprinted polymer (MIP) is another option, achieving a low LOD of 0.01 pg/μL (=10 ppt). LFIAs have successfully been used for the sensitive and specific detection of various pathogens using a target-specific antibody. However, PFASs (including PFOA), currently do not have a known antibody that could be used; therefore, this popular method is not applicable.

Accordingly, there is a need for better methods, compositions, systems apparatus, and devices that utilize a biomolecule to specifically detect an environmental toxicant by utilizing competitive interactions during capillary action.

### SUMMARY

Disclosed herein is a composition for detecting one or more environmental toxicants in a liquid sample by decreasing the flow of the liquid sample comprising one or more environmental toxicants comprising: one or more cellulose fibers; and one or more biomolecules associated with the one or more cellulose fibers, wherein the composition is configured so that when the composition contacts the liquid sample, the one or more biomolecules disassociate from the one or more cellulose fibers by interacting with the one or more environmental toxicants thereby decreasing the flow of the liquid sample comprising one or more environmental toxicants. Aspects disclosed herein, can detect perfluorinated-alkyl substance (PFAS) such as, but not limited to, perfluorooctanoic acid (PFOA). In some aspects, the one or more cellulose fibers associate with the one or more biomolecules by mechanically capturing the biomolecule within the one or more cellulose fibers. In particular aspects disclosed herein, the one or more biomolecules interact with the one or more environmental toxicants by charge interaction, hydrophilic interactions, hydrophobic interactions, affinity interactions, hydrogen bonding, electrostatic repulsion, electrostatic attraction, Van der Waals forces, or any combination thereof.

In aspects disclosed herein, the one or more biomolecules comprise a first biomolecule, a second biomolecule that is different from the first biomolecule, and a third biomolecule that is different from the first biomolecule and the second biomolecule. The one or more biomolecules can be an amino acid, a polypeptide, a protein, or any combination thereof. In particular aspects disclosed herein, the one or more biomolecules is L-lysine, bovine serum albumin (BSA), casein, or any combination thereof.

Also disclosed herein is a paper microfluidic chip, for detecting one or more environmental toxicants, comprising: a paper substrate comprising one or more cellulosic fibers having a first side and a second side; one or more biomolecules associated with the one or more cellulosic fibers; and an inlet for receiving a liquid sample comprising one or more environmental toxicants, wherein the inlet is in communication with one or more microchannels defined by one or more walls, and wherein the walls that define the at least one microchannel extend through the paper substrate from the first side to the second side thereby defining one or more microchannels in the paper substrate. Also disclosed herein is a system, comprising: the paper microfluidic chip disclosed herein, an image capture device; and a computer program for analyzing the data collected by the image capture device.

Disclosed herein is a method for detecting one or more environmental toxicants in a liquid sample, comprising: providing the liquid sample to the inlet of the paper microfluidic chip according to claim 10; measuring the flow distance of the liquid sample along the one or more microchannels; and analyzing a flow profile, wherein the flow profile comprises a plurality of measured flow distances against time.

Also disclosed herein is a method for making the paper microfluidic chip disclosed herein, comprising: using a wax printer to print wax walls onto a cellulose paper, the wax walls defining at least one microfluidic channel on the cellulose paper; heating the cellulose paper at a temperature sufficient to melt the wax into the cellulose paper such that the wax walls extend through the cellulose paper from a first side to a second side of the cellulose paper, thereby forming a microchannel in the cellulose paper defined by the wax walls; and loading a biomolecule into the microchannel and thereby associate the one or more biomolecules with one or more cellulose fibers of the paper microfluidic chip.

These and other features of the present disclosure will become apparent from the detailed description in sections below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing of an aspect of the composition disclosed herein depicting molecular interactions of a cellulose fiber, a biomolecule, and a target molecule.

FIG. 2 depicts a planar view of an aspect of a paper microfluidic chip disclosed herein.

FIG. 3 is a schematic drawing of an aspect of the composition disclosed herein depicting molecular interactions between one embodiment of the cellulose fibers, biomolecule, and target molecule.

FIG. 4A depicts a perspective view of an aspect of the system disclosed herein for detecting an environmental toxicant.

FIGS. 4B-4E depicts a perspective view of an exemplary aspect of the system method disclosed herein for detecting PFOA.

FIGS. 5A-5D show the flow rate profiles of a microfluidic chip with no reagent and a sample comprising different concentrations of PFOA showing flow distance (pixels) versus time (30 frame/s).

FIGS. 6A-6D show the flow rate profiles of a microfluidic chip comprising 10 ng/μL L-lysine and a sample comprising different concentrations of PFOA showing flow distance (pixels) versus time (30 frame/s).

FIGS. 7A-7D show the flow rate profiles of a microfluidic chip comprising 0.001 ng/μL L-lysine and a sample comprising different concentrations of PFOA showing flow distance (pixels) versus time (30 frame/s).

FIGS. 8A-8D show the flow rate profiles of a microfluidic chip comprising 10 ng/μL casein and a sample comprising different concentrations of PFOA showing flow distance (pixels) versus time (30 frame/s).

FIGS. 9A-9D show the flow rate profiles of a microfluidic chip comprising 0.001 ng/μL casein and a sample comprising different concentrations of PFOA showing flow distance (pixels) versus time (30 frame/s).

FIGS. 10A-10D show the flow rate profiles of a microfluidic chip comprising 10 ng/μL BSA and a sample comprising different concentrations of PFOA showing flow distance (pixels) versus time (30 frame/s).

FIGS. 11A-11D show the flow rate profiles of a microfluidic chip comprising 0.001 ng/μL BSA and a sample comprising different concentrations of PFOA showing flow distance (pixels) versus time (30 frame/s).

FIG. 12 depicts a bar graph of flow distance (pixels) versus preloaded reagent (ng/μL).

FIG. 13 depicts a bar graph of flow distance (pixels) versus PFOA concentration (fg/μL).

FIG. 14A depicts a bar comparing normalized flow distance versus spiked PFOA concentration (fg/μL) for influent wastewater using a microfluidic chip comprising 10 ng/μL BSA.

FIG. 14B depicts a bar comparing normalized flow distance versus spiked PFOA concentration (fg/μL) for effluent wastewater using a microfluidic chip comprising 10 ng/μL BSA.

FIGS. 15A-15D show the flow rate profiles of a microfluidic chip comprising no preloaded biomolecule and a sample comprising deionized water and different concentrations of PFOA showing flow distance (pixels) versus time (30 frame/s).

FIG. 16A-16C show the flow rate profiles of a microfluidic chip comprising no biomolecule and a sample comprising different concentrations of SDS showing flow distance (pixels) versus time (30 frame/s).

FIGS. 17A-17C show the flow rate profiles of a microfluidic chip comprising no biomolecule and a sample comprising different concentrations of TWEEN showing flow distance (pixels) versus time (30 frame/s).

FIGS. 18A-18C show the flow rate profiles of a microfluidic chip comprising no biomolecule and a sample comprising different concentrations of CTAB showing flow distance (pixels) versus time (30 frame/s).

FIGS. 19A-19D show the flow rate profiles of a microfluidic chip comprising 10 ng/μL L-lysine and a sample comprising different concentrations of PFOA showing flow distance (pixels) versus time (30 frame/s).

FIGS. 20A-20C show the flow rate profiles of a microfluidic chip comprising 10 ng/μL L-lysine and a sample comprising different concentrations of SDS showing flow distance (pixels) versus time (30 frame/s).

FIGS. 21A-21C show the flow rate profiles of a microfluidic chip comprising 10 ng/μL L-lysine and a sample comprising different concentrations of TWEEN showing flow distance (pixels) versus time (30 frame/s).

FIGS. 22A-22C show the flow rate profiles of a microfluidic chip comprising 10 ng/μL L-lysine and a sample comprising different concentrations of CTAB showing flow distance (pixels) versus time (30 frame/s).

FIGS. 23A-23D show the flow rate profiles of a microfluidic chip comprising 0.001 ng/μL L-lysine and a sample comprising different concentrations of PFOA showing flow distance (pixels) versus time (30 frame/s).

FIGS. 24A-24C show the flow rate profiles of a microfluidic chip comprising 0.001 ng/μL L-lysine and a sample comprising different concentrations of SDS showing flow distance (pixels) versus time (30 frame/s).

FIGS. 25A-25C show the flow rate profiles of a microfluidic chip comprising 0.001 ng/μL L-lysine and a sample comprising different concentrations of TWEEN showing flow distance (pixels) versus time (30 frame/s).

FIGS. 26A-26C show the flow rate profiles of a microfluidic chip comprising 0.001 ng/μL L-lysine and a sample comprising different concentrations of CTAB showing flow distance (pixels) versus time (30 frame/s).

FIGS. 27A-27D show the flow rate profiles of a microfluidic chip comprising 10 ng/μL casein and a sample comprising different concentrations of PFOA showing flow distance (pixels) versus time (30 frame/s).

FIGS. 28A-28C show the flow rate profiles of a microfluidic chip comprising 10 ng/μL casein and a sample comprising different concentrations of SDS showing flow distance (pixels) versus time (30 frame/s).

FIGS. 29A-29C show the flow rate profiles of a microfluidic chip comprising 10 ng/μL casein and a sample comprising different concentrations of TWEEN showing flow distance (pixels) versus time (30 frame/s).

FIGS. 30A-30C show the flow rate profiles of a microfluidic chip comprising 10 ng/μL L-casein and a sample comprising different concentrations of CTAB showing flow distance (pixels) versus time (30 frame/s).

FIGS. 31A-31D show the flow rate profiles of a microfluidic chip comprising 0.001 ng/μL casein and a sample comprising different concentrations of PFOA showing flow distance (pixels) versus time (30 frame/s).

FIGS. 32A-32C show the flow rate profiles of a microfluidic chip comprising 0.001 ng/μL casein and a sample comprising different concentrations of SDS showing flow distance (pixels) versus time (30 frame/s).

FIGS. 33A-33C show the flow rate profiles of a microfluidic chip comprising 0.001 ng/μL casein and a sample comprising different concentrations of TWEEN showing flow distance (pixels) versus time (30 frame/s).

FIGS. 34A-34C show the flow rate profiles of a microfluidic chip comprising 0.001 ng/μL casein and a sample comprising different concentrations of CTAB showing flow distance (pixels) versus time (30 frame/s).

FIGS. 35A-35D show the flow rate profiles of a microfluidic chip comprising 10 ng/μL BSA and a sample comprising different concentrations of PFOA showing flow distance (pixels) versus time (30 frame/s).

FIGS. 36A-36C show the flow rate profiles of a microfluidic chip comprising 10 ng/μL BSA and a sample comprising different concentrations of SDS showing flow distance (pixels) versus time (30 frame/s).

FIGS. 37A-37C show the flow rate profiles of a microfluidic chip comprising 10 ng/μL BSA and a sample comprising different concentrations of TWEEN showing flow distance (pixels) versus time (30 frame/s).

FIGS. 38A-38C show the flow rate profiles of a microfluidic chip comprising 10 ng/μL BSA and a sample comprising different concentrations of CTAB showing flow distance (pixels) versus time (30 frame/s).

FIGS. 39A-39D show the flow rate profiles of a microfluidic chip comprising 0.001 ng/μL BSA and a sample comprising different concentrations of PFOA showing flow distance (pixels) versus time (30 frame/s).

FIGS. 40A-40C show the flow rate profiles of a microfluidic chip comprising 0.001 ng/μL BSA and a sample comprising different concentrations of SDS showing flow distance (pixels) versus time (30 frame/s).

FIGS. 41A-41C show the flow rate profiles of a microfluidic chip comprising 0.001 ng/μL BSA and a sample comprising different concentrations of TWEEN showing flow distance (pixels) versus time (30 frame/s).

FIGS. 42A-42C show the flow rate profiles of a microfluidic chip comprising 0.001 ng/μL BSA and a sample comprising different concentrations of CTAB showing flow distance (pixels) versus time (30 frame/s).

FIG. 43 depicts a bar graph of flow distance (pixels) versus pre-loaded reagent (ng/μL) showing the specificity of 0.001 ng/μL L-lysin, 10 ng/μL L-lysine, 0.001 ng/μL casein, 10 ng/μL casein, 0.001 ng/μL BSA, 10 ng/μL BSA to other non-fluorocarbon surfactants (e.g., anionic sodium dodecyl sulfate (SDS), non-ionic Tween 20, and cationic cetrimonium bromide (CTAB)).

FIG. 44 is a schematic drawing depicting the results of a protocol for distinguishing PFOA and non-fluorocarbon surfactants.

### DETAILED DESCRIPTION

### I. Abbreviations

BSA: Bovine serum albumin
CTAB: Cetrimonium bromide
PFAS: Perfluorinated-alkyl substance
PFOA: Perfluorooctanoic acid
SDS: Sodium dodecyl sulfate

### II. Overview of Terms, Ranges, and Definitions

Both the foregoing general description and the following detailed description are exemplary and explanatory only and are not intended to limit the scope of the present disclosure.

As used herein, the use of the singular includes the plural unless specifically stated otherwise. For example, the singular forms “a”, “an” and “the” as used in the specification also include plural aspects unless the context dictates otherwise. Similarly, any singular term used in the specification also means plural or vice versa, unless the context dictates otherwise.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

The term “or combinations thereof” as used herein refers to all permutations and combinations of the listed items preceding the term. For example, “A, B, C, or combinations thereof” is intended to include at least: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth.

Amino Acid: An organic acid containing both a basic amino group (—NH₂) and an acidic carboxyl group (—COOH). The 25 amino acids that are protein constituents are α-amino acids, i.e., the —NH₂ group is attached to the carbon atom next to the —COOH group.

Biomolecule: Any molecule that may be included in a biological system, including but not limited to, a synthetic or naturally occurring protein, glycoprotein, lipoprotein, amino acid, nucleoside, nucleotide, nucleic acid, oligonucleotide, DNA, RNA, carbohydrate, sugar, lipid, fatty acid, hapten, and the like.

Cellulose: A naturally occurring polysaccharide of about 70 to more than 10,000 β(1→4) linked D-glucose units in a linear chain and has the general formula (C₆H₁₀O₅)ₙ. Cellulose is a structural component of plant cell walls. About one third of plant matter is cellulose. Wood contains approximately 50% cellulose by weight. Cellulose polymers can be characterized by the degree of polymerization (dp), which is the number of monomer units, i.e., glucose units. Cellulose polymers may contain from several hundred to several thousand glucose units. For example, the degree of polymerization can range from about 1000 for wood pulp to about 3500 for cotton fiber. Cellulose can be decomposed into glucose by hydrolysis or by the enzyme cellulase.

Control: A sample or procedure performed to assess test validity. In one example, a control is a positive control, which comprises the actual target sample. A negative control comprises a sample similar to the positive control but which is known from previous experience to give a positive result. A negative control confirms that the basic conditions of the test produce a positive result.

Polypeptide: A polymer in which the monomers are amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

Sample: The term “sample” refers to any liquid, semi-solid or solid substance (or material) in or on which a target can be present.

Surfactant/surface active material: A compound that reduces surface tension when dissolved in water or water solutions, or that reduces interfacial tension between two liquids. A surfactant molecule typically has a polar or ionic “head” and a nonpolar hydrocarbon “tail.”

All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, the definitions provided by this specification control. While the present teachings are described in conjunction with various embodiments, it is not intended that the present teachings be limited to such embodiments. On the contrary, the present teachings encompass various alternatives, modifications, and equivalents, as will be appreciated by a person of ordinary skill in the art in light of the present teachings.

### III. Composition

Aspects of the present disclosure are directed to a composition for detecting one or more environmental toxicants in a liquid sample contained therein. In some aspects of the present disclosure, the composition comprises one or more cellulose fibers; and one or more biomolecules associated with the one or more cellulose fibers, wherein the composition is configured such that when the composition contacts the liquid sample, the one or more biomolecules disassociate from the one or more cellulose fibers by interacting with the one or more environmental toxicants thereby decreasing the flow of the liquid sample comprising one or more environmental toxicants. Moreover, the biomolecules are not covalently bound to the environmental toxicants or the cellulose fibers simplifying the assays. In some aspects, the composition disclosed herein has an environmental toxicant level of detection less than 70 ppt (70 fg/μg).

In particular aspects of the present disclosure, the one or more biomolecules can interact with the one or more environmental toxicants by charge interaction, hydrophilic interactions, hydrophobic interactions, affinity interactions, hydrogen bonding, electrostatic repulsion, electrostatic attraction or any combination thereof. Accordingly, these interactions can release the one or more biomolecule from the one or more cellulose fibers and thereby interact with the one or more environmental toxicants, and subsequently lower the capillary flow rate of the liquid sample comprising the one or more environmental toxicants. In some aspects, the liquid sample comprising an environmental toxicant is applied to the composition disclosed herein, wherein the released environmental toxicant molecules are adsorbed to the wetting front, i.e., liquid-gas interface, lowering the surface tension and subsequently the capillary flow rate, which can decreases the flow rate of the liquid sample comprising one or more environmental toxicants.

In particular aspects of the present disclosure, the one or more biomolecules of the composition disclosed herein can associate with the cellulose fibers of nitrocellulose or cellulose. Without being limited to a single theory, the one or more cellulose fibers, which are not charged per se, but are polar and can allow water molecules to be absorbed easily within the fibers via hydrogen bonds between the one or more environmental toxicants and the one or more biomolecules. In some aspects, the one or more cellulose fibers can carry a weak charge upon carboxylation, which can allow for electrostatic attraction or repulsion with one or more environmental toxicants and the one or more biomolecules. However, PFOA is unlikely to interact with one or more cellulose fibers because of its hydrophobicity (cellulose is strongly hydrophilic) and its negative charge/dipole.

In some aspects, the composition disclosed herein may comprise a combination of at least one biomolecule that can interact with the one or more environmental toxicants with sufficiently strong hydrophobic interactions, electrostatic attractions, hydrogen bonding, or any combination thereof that release the one or more biomolecules from the cellulose fibers and thereby can disassociate the one or more biomolecule from one or more cellulose fibers. The present compositions therefore provide for the detection of an environmental toxicant by analyzing the capillary flow rate of a liquid sample to identify the presence of a target environmental toxicant based on the molecular interactions. The inventors have found that the present compositions provide unexpectedly rapid, economical, and efficient approaches in detecting the presence of environmental toxicants.

The environmental toxicant can be any perfluorinated-alkyl substance such as, but not limited to perfluorooctanoic acid (PFOA). PFOA can interact with a biomolecule disclosed herein by hydrophobic interactions, electrostatic interactions (including attraction and repulsion), and hydrogen bonding. The extreme hydrophobicity of the fluorocarbon tail of PFOA is unique, even when compared to the hydrocarbon tail present on other non-fluorocarbon surfactants.

In particular aspects disclosed herein, the one or more biomolecules such as, but not limited to an amino acid, a polypeptide, or a protein, or any combination thereof, can be used. In some aspects, the one or more biomolecules can be an amino acid. In particular aspects of the present disclosure, the biomolecule can be an amino acid having a positive charge under physiological pH, which would enable the amino acid to associate with the cellulose fibers without being mechanically captured by the cellulose fibers. In preferable aspects, the one or more biomolecules may comprise positively charged side chains. In more preferable aspects, the one or more biomolecules can be L-lysine, histidine, arginine, glutamic acid, or any combination thereof. In a preferred example, the one or more biomolecules can be an amino acid such as, but not limited to L-lysine.

Without being limited to a single theory, proteins disclosed herein may carry a net negative charge under physiological pH and might be repelled from the cellulose fibers with a weak negative charge or dipole. However, their high molecular weight compared to PFOA, amino acids, and other non-fluorocarbon surfactants can enable them to associate with the one or more cellulose fibers. For example, the proteins can be mechanically captured within the fibers, utilizing the local positive charge/dipoles on their surfaces.

In aspects of the present disclosure, the protein can be a globular protein having a molecular weight in the range of 15 kDa to 100 kDa, such as from 15 kDa to 80 kDa, or from 15 kDa to 70 kDa. In some aspects, the globular protein may have hydrophobic properties, wherein the water contact angle can be from 50° to 100°, such as from 50° to 80°, or preferably from 55° to 75″. Without being limited to a single theory, such strong hydrophobicity may enable hydrophobic interactions with PFOA.

In particular aspects disclosed herein, the globular protein may comprise an electric point of from 3 to 6, such as from 4 to 6, or preferably from 4 to 5, rendering its net charge to negative under physiological pH. However, a net negative charge does not imply that the entire globular protein comprises a negative charge because portions of the globular protein may still carry a positive charge under physiological pH and thus allowing the globular protein to interact with the carboxyl group of PFOA.

In one preferred example, the globular protein is an albumin such as, but not limited to bovine serum albumin. Without being limited to a single theory, when PFOA and BSA are present within the cellulose fibers, different forms of hydrogen bonds can form among PFOA, BSA, and the cellulose fibers because cellulose fibers are not charged per se-they are still polar-allowing water molecules to be absorbed within the fibers. Additionally, cellulose fibers can carry a weak charge upon carboxylation, which can allow weak electrostatic attraction or repulsion. However, PFOA can still interact with BSA via hydrophobic interactions, electrostatic interactions, and hydrogen bonding.

Although BSA is highly non-specific, it has both hydrophobic and hydrophilic sections that can interact specifically with PFOA. In addition, the competitive interactions with cellulose fibers might also contribute to the specificity of PFOA. If these interactions are more substantial than the BSA-cellulose interactions, BSA may leave the cellulose fibers after passive immobilization. Moreover, PFOA may not interact with cellulose fibers, considering its hydrophobicity (cellulose is strongly hydrophilic) and the negative charge/dipole (cellulose fibers also carry negative charge/dipole). Thus, since BSA carries a net negative charge under physiological pH, it may be repelled from the cellulose fibers with a weak negative charge or dipole. However, BSA's high molecular weight (compared to PFOA and other non-fluorocarbon surfactants) may enable BSA to associate with cellulose fibers by being mechanically captured within the fibers by passive immobilization, utilizing the local positive charge/dipoles on their surfaces.

In another preferred example, the globular protein can be casein. Similar to BSA, casein also comprises a low isoelectric point (4.6). However, casein can behave differently from BSA, considering its smaller molecular weight (20-25 kDa casein vs. 66 kDa BSA). Without being limited by a single theory, the cellulose fibers can repel casein because it carries a net negative charge under physiological pH; however, casein can be mechanically captured within the fibers by utilizing the local positive charge/dipoles on its surface.

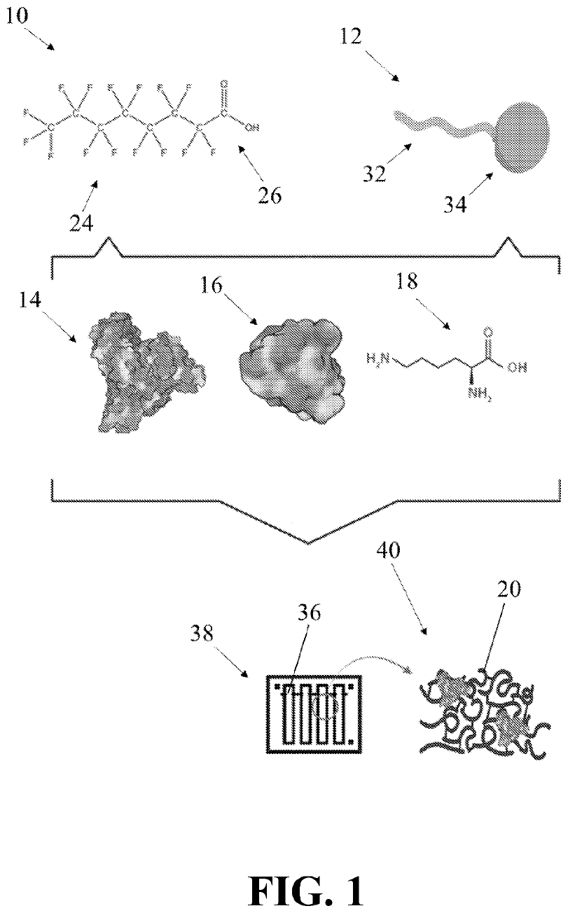

FIG. 1 is a non-limiting schematic illustrating molecular interactions between, PFOA 10, non-fluorocarbon surfactants 12, BSA 14, casein 16, L-lysine 18, and the cellulose fibers 20 of a paper microfluidic chip 22. Specifically, PFOA 10 can interact with the one or more biomolecules via hydrophobic interactions with the hydrophobic tail 24 of the PFOA 10 and by hydrogen bonding and electrostatic interactions with the hydrophilic head/negative dipole 26 on PFOA 10. Non-fluorocarbon surfactants 12 (e.g., SDS (−), CTAB (+), and TWEEN (nonionic)) can potentially interact with one or more biomolecules via hydrophobic interactions on the hydrophobic hydrocarbon tail 32 and the electrostatic interactions on the functional head 34, which gives the surfactant a charge. The biomolecules can associate with cellulose fibers 20 to different degrees (i.e., strengths), e.g., hydrophobic interactions electrostatic attraction, electrostatic repulsion, hydrogen bonding, mechanical capturing, passive immobilization on the channels 36 of the cellulose microfluidic chip 38. The blue shaded regions are hydrophobic portions, the red shaded regions are the hydrophilic regions of the biomolecules. Both BSA 14 and casein 16 carry a net negative charge under physiological pH and might be repelled from the cellulose fibers 20 with a weak negative charge or dipole. However, BSA 14 and casein 16 have a higher molecular weight compared to PFOA 10, L-lysine 18, and other surfactants 12, which would promote the mechanical capture 40 of BSA 14 and casein 16 within the cellulose fibers 20 by utilizing the local positive charge/dipoles on their surfaces as shown in. With reference to L-lysine 18, the two aminos may have a positive charge or dipole and oxygens on the carboxylic acid may have a negative charge or dipole. Thus, L-lysine 18 can associate with cellulose fibers 20 via the positive charge under physiological pH without being mechanically captured.

In some aspects, the composition disclosed herein can be used to detect greater than 0.05 fg/μL environmental toxicants in a sample. For example, the PFOA limit of detection has a range greater than 0.05 fg/μL, such as from 0.05 fg/μL to 5000 fg/μL, preferably from 0.05 fg/μL to 2500 fg/μL, most preferably from 1 fg/μL to 1000 fg/μL.

In particular aspect disclosed herein, the concentration of the one or more biomolecules has a range from 0.0005 ng/μL to 25 ng/μL. In preferable aspects, the one or more biomolecule can have a concentration in the range of from 0.0005 ng/μL to 15 ng/μL. In most preferable aspect, the biomolecule can have a concentration in the range of 0.001 ng/μL to 10 ng/μL. In one example, the composition comprises 0.001 ng/μL L-lysine. In another example, the composition comprises 10 ng/μL L-lysine. In another example, the composition comprises 0.001 ng/μL casein. In yet another example, the composition comprises 10 ng/μL casein. In one example, the composition comprises 0.001 ng/μL BSA. In yet another example, the composition comprises 10 ng/μL BSA. In a preferable example, the composition comprises 0.001 ng/μL L-lysine and 10 ng/μL BSA.

### IV. Paper Microfluidic Chip

Aspects of the present disclosure are directed to a paper microfluidic chip for detecting environmental toxicants. The paper microfluidic chip can have a first side and a second side and one or more walls that define one or more microchannels and extend through the paper substrate from the first side to the second side. The paper microfluidic chip may also comprise an inlet that is in communication with one or more microchannels. The inlet of the microfluidic channel can be configured for receiving a sample comprising one or more environmental toxicants such as, but not limited to, PFOA.

In particular aspects disclosed herein, the one or more channels can have a width from 0.5 mm to 10 mm, preferably from 1 mm to 8 mm, and most preferably 1 to 5 mm. in some aspects, the paper thickness of the microfluidic chip can range from 50 μm to 500 μm, preferably from 50 μm to 350 μm, most preferably from 100 μm to 200 μm.

In some aspects, the paper substrate can be cellulose, nitrocellulose, or a combination thereof. The cellulose fibers of the cellulose or nitrocellulose can associate with one or more biomolecules. In particular aspects disclosed herein, one or more biomolecules can be provided between the inlet and halfway the length of the microchannel. For example, one or more biomolecules such as, but not limited to, an amino acid, a polypeptide, or a protein, can be immobilized on the paper microfluidic chip between the inlet and halfway the length of the microchannel such the one or more biomolecules associate with one or more cellulose fibers of the paper substrate.

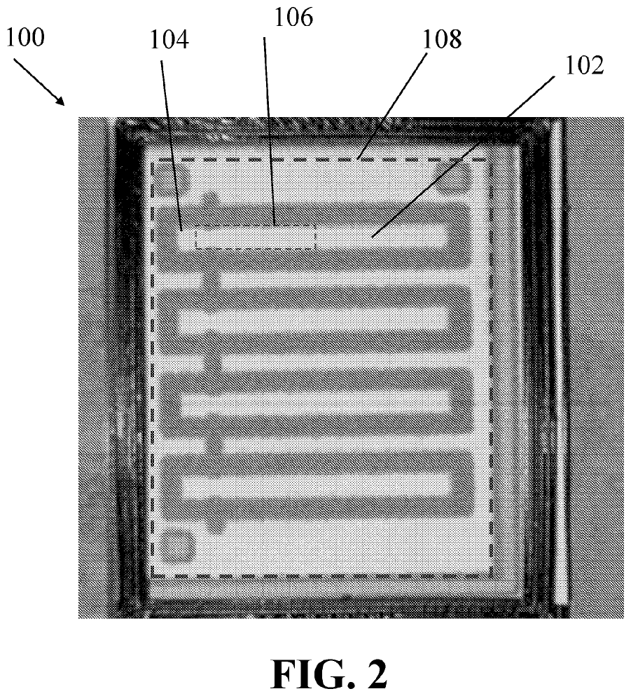

FIG. 2 illustrates an exemplary paper microfluidic chip 100 comprising four microfluidic channels 102, sample inlet 104, an immobilized area 106 for a biomolecule, and a detection zone 108.

In aspects of the present disclosure, the protein can be a globular protein having a molecular weight in the range of 15 kDa to 100 kDa, such as from 15 kDa to 80 kDa, or from 15 kDa to 70 kDa. In some aspects, the globular protein may have hydrophobic properties, wherein the water contact angle can be from 50° to 100°, such as from 50° to 80°, or preferably from 55° to 75°.

Without being limited to a single theory, such strong hydrophobicity may enable hydrophobic interactions with PFOA. In particular aspects disclosed herein, the globular protein may comprise an electric point from 3 to 6, such as from 4 to 6, or preferably from 4 to 5, rendering its net charge to negative under physiological pH. However, a net negative charge does not imply that the entire globular protein comprises a negative charge because portions of the globular protein may still carry a positive charge under physiological pH and thus allowing the globular protein to interact with the carboxyl group of PFOA. In one preferred example, the globular protein is an albumin such as, but not limited to bovine serum albumin. In another preferred example, the globular protein is casein.

In some aspects, the paper microfluidic chip is preloaded with one or more biomolecules such; thus, a reagent comprising one or more biomolecules is provided and allowed to dry so that the one or more biomolecules can associate with the one or more biomolecules, e.g., BSA is mechanically captured or L-lysine is captured electrostatically. When the one or more biomolecules are not preloaded, some of the biomolecules may not associate with the cellulose fibers and can be found at the wetting front i.e., the water-air interface, and thus the capillary flow rate becomes closer to water.

In particular aspect disclosed herein, the one or more biomolecules preloaded can have a concentration in the range from 0.0005 ng/μL to 25 ng/μL. In preferable aspects, the biomolecule can have a concentration in the range of from 0.0005 ng/μL to 15 ng/μL. In most preferable aspect, the biomolecule can have a concentration in the range of 0.001 ng/μL to 10 ng/μL. In one example, 0.001 ng/μL L-lysine is preloaded on the microfluidic paper chip. In another example, 10 ng/μL L-lysine is preloaded on the microfluidic paper chip. In another example, 0.001 ng/μL casein is preloaded on the microfluidic paper chip. In yet another example, 10 ng/μL casein is preloaded on the microfluidic paper chip. In one example, 0.001 ng/μL BSA is preloaded on the microfluidic paper chip. In yet another example, 10 ng/μL BSA is preloaded on the microfluidic paper chip. In a preferable example, 0.001 ng/μL L-lysine and 10 ng/μL BSA are preloaded on the microfluidic paper chip.

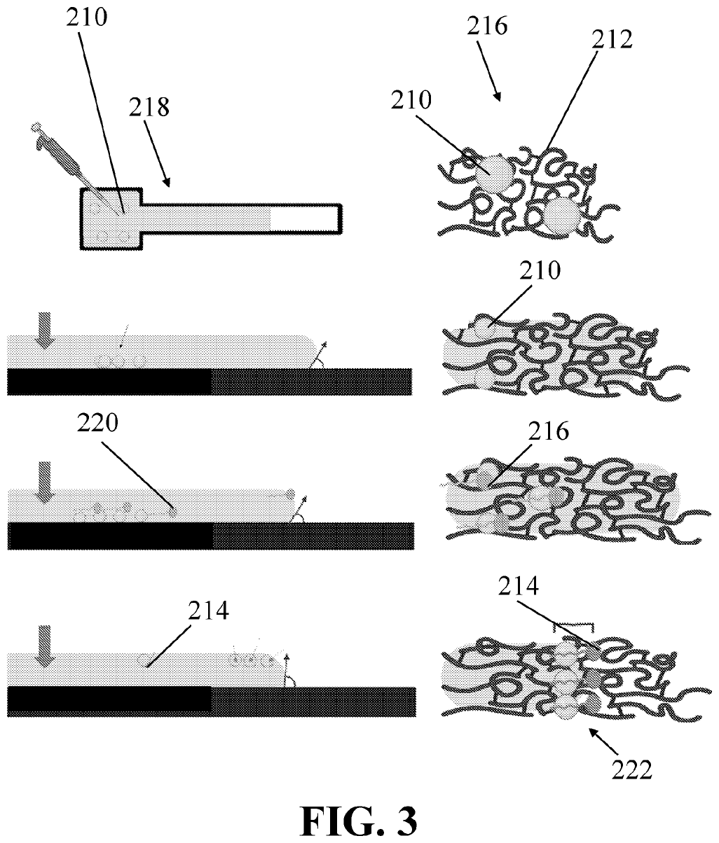

FIG. 3 is non-limiting schematic illustrating how the molecular interactions between preloaded BSA 210, the cellulose fibers 212 of the paper substrate, and PFOA 214 in a sample can affect the interfacial tension and subsequently the capillary flow rate. The preloaded BSA 210 allows for the one or more cellulose fibers 212 to associate with the BSA 210, wherein the cellulose fibers 212 mechanically capture 216 the BSA 210. A sample comprising only DI water 218 and no PFOA 214 results in high surface tension and subsequently faster flow. A sample comprising Tween 220 (i.e., negative control) results in the wetting front comprising of some tween with high surface tension and hence faster flow because the interactions of the Tween 220 and BSA 210 are not strong enough to disassociate the BSA 210 from the one or more cellulose fibers 212. A sample comprising PFOA 214 (i.e., positive control) results in the wetting front comprising one more PFOA-BSA conjugates 222 with low surface tension and hence slower flow because PFOA 214 binds to BSA 210 with multiple interactions (such as but not limited to hydrophobic interactions, electrostatic interactions, and hydrogen bonding) and are strong enough to interact with PFOA 214 such that the one or more biomolecules disassociate from the one or more cellulose fibers.

### V. System

Also disclosed herein is a system for detecting an environmental toxicant comprising the paper microfluidic chip disclosed herein; an image capture device; and a computer program for analyzing the data collected by the image capture device. In some aspects, the paper microfluidic chip may comprise a detection zone. The detection zone area continuously monitors the capillary flow rate. In one example, the detection zone area may comprise one or more biomolecule such as, but are not limited to L-lysine, casein, BSA, or any combination thereof. In a preferred example, the one or more biomolecules are pre-immobilized onto the microfluidic channel and captured within the cellulose fibers mechanically and/or electrostatically. In the presence of PFOA, PFOA and the one or more biomolecules interact by, but are not limited to, hydrophobic interactions, electrostatic attractions, and/or hydrogen bonding. Such strong interactions dissociate release the one or more biomolecules from one or more cellulose fibers of the paper substrate to form to PFOA-biomolecule conjugates. The released PFOA-reagent conjugates are adsorbed at the wetting front, i.e., liquid-gas interface, lowering the surface tension and subsequently the capillary flow rate. In some aspects, the decrease in flow rate in comparison to the flow rate of a control sample is used to identify the presence of PFOA in a sample.

The system for detecting a perfluorooctanoic acid (PFOA) can include a computer program for analyzing data collected by the image capture device. In some aspects, the data is uploaded to a remote cloud-based storage from an image capture device and automatically analyzed using a computer code. The computer code can be preset to recognize the channel layout and wetting front and thereby evaluate the flow distances in pixels to generate a computer file comprising cumulative data of the flow distance over time frame. For example, video clips can be uploaded to Google Drive from an image capture device and analyzed using a preset Python code in Google Colab, thereby collecting a raw flow rate profile for each microfluidic channel on the cellulosic microfluidic chip.

In a particular aspects disclosed herein, the image capture device is a smartphone that includes a video recording function. In some aspects, the smartphone may include a flash function and grid view function for recording a video. In some aspects, the system may further comprise a platform for securing the image capture device. In some aspects, the system for detecting a perfluorooctanoic acid (PFOA) includes a chip holder for securing the cellulosic microfluidic chip.

### VI. Method

The present disclosure also provides for a method for detecting a perfluorooctanoic acid (PFOA) by providing a liquid sample comprising one or more environmental toxicants disclosed herein to the inlet of the paper microfluidic chip; measuring the flow distance of the liquid sample along one or more channels; and analyzing a flow profile, wherein the flow profile comprises a plurality of measured flow distances against time.

In some aspects, the method can detect greater than 0.05 fg/μL environmental toxicants in a sample. For example, the PFOA limit of detection has a range greater than 0.05 fg/μL, such as from 0.05 fg/μL to 5000 fg/μL, preferably from 0.05 fg/μL to 2500 fg/L, most preferably from 1 fg/μL to 1000 fg/μL.

In some aspects, the method can further comprise providing an image capture device for measuring the flow distance in pixels and the time in frames. For example, a video clip can be recorded via the image capture device under ambient lighting. In another example the method does not require a separate light source, optical filters, or a dark environment.

While non-limiting, the following simple representative calculations can be used to illustrate the performance of some aspects of the present disclosure. In aspects of the present disclosure, the video clip can be delivered to a computer program to identify a “flow front” in an automated manner by plotting the flow distance (L) over (t) to generate a capillary flow profile that fits the Lucas-Washburne equation:

$$\frac{{L}^{2}}{t} = d γcosθ / 2 μ$$

where d is the pore size, γ is the liquid-vapor interfacial tension, θ is water contact angle, and μ is the viscosity of liquid. The equation can be simplified to:

$$L = {A ( t - b )}^{1 / 2}$$

where A is the lumped parameter representing the interfacial tension and viscosity, and b represent the time offset (i.e., adjusting the time where the flow starts), and wherein the lumped parameter A was used as a sensor readout. The biomolecule such as, but not limited to, L-lysine, casein, or BSA according to the present disclosure associate with the cellulose fibers in a non-specific manner when no PFOA is present. However, upon the presence of one or more environmental toxicants such as, but not limited to PFOA, the biomolecules can interact with PFOA and thus are no longer associated with the cellulose fibers and continue to flow. These free-flowing PFOA-biomolecule conjugates components can lower the surface tension and, subsequently, the lumped parameter A. In view of this, parameter A should decrease with increasing PFOA concentration. If the target PFOA concentration is substantially higher than the PFOA conjugates, either the interaction is not preferred, or the high PFOA concentration lowers the overall surface tension, leading to the increase in A. Therefore, in some examples, the shape of the “standard curve” forms an upside-down bell shape.

In particular aspects disclosed herein, information obtained by the image capture device can be delivered to a remote cloud-based storage and analyzing using computer programmable code recognizes the channel layout and the wetting front to evaluate the flow distances in pixels. Accordingly, cumulative data of the flow distances over the time frame such as but not limited to 30 frames per second is generated. For example, videos can be uploaded to Google Drive and analyzed using a computer programmable code hosted in Google Colab, wherein the computer programmable code such as, but not limited to, Python code automatically recognizes the channel layout and the wetting front to evaluate the flow distances to generate cumulative data of the flow distance over the time frame.

In some aspects, data with statistically significant differences between positive and negative controls can be generated, such as, but not limited to, as early as 5 seconds or as late as 30 seconds, depending on the biomolecule used. The flow distances can be collected at such time, such as, but not limited to, 5 s, 10 s, 20 s, or 30 s, by taking the averages from 30 frames (1 s), such as, but not limited to, 20 s to 21 s, which can be one signal from a specific channel. This can be repeated multiple times using different microfluidic channels, and the averages and standard errors are evaluated. In some examples, the entire flow profile, which is the cumulative data set of the flow distances (in pixels) against time (in frames) can be analyzed by fitting the data to a square root curve, following the Lucas-Washburn equation, which can be rearranged to:

$$L = \sqrt{\frac{tR {γ}_{LG} cos θ}{2 μ}}$$

where: L is flow distance, t is time, R is the capillary radius, γ_{LG} is surface tension at the liquid-gas interface, θ is water contact angle, and u is dynamic viscosity. In one example, R, γ_{LG}, θ, and μ are assumed to not change during the flow (i.e., remain constant), and hence the flow distance L becomes a function of a square room of time t. Furthermore, the square root curves can be fitted and plotted in a computer program. For example, square root curves can be fitted in Origin (OriginLab; Northampton, MA, USA) and plotted in Microsoft Excel.

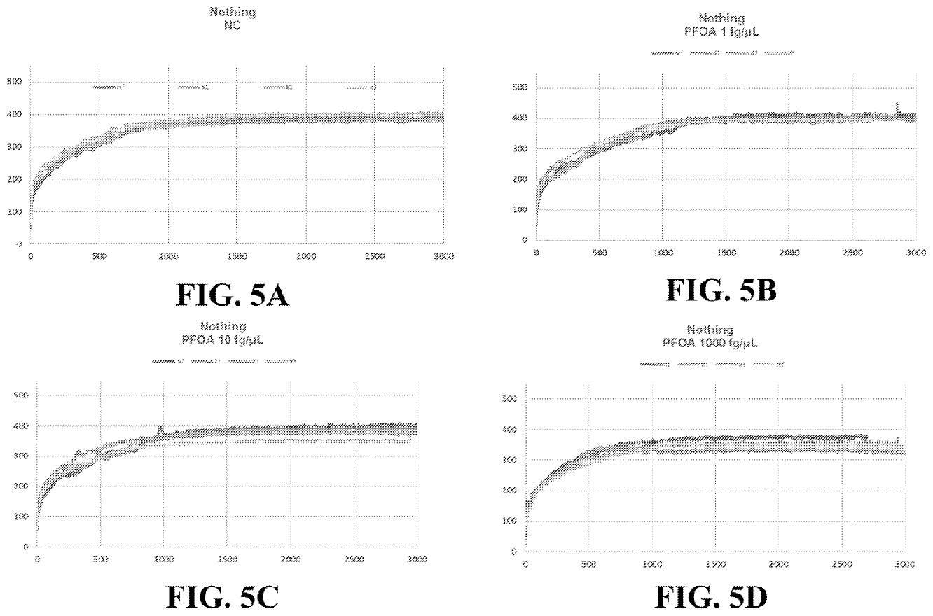

In particular aspects disclosed herein, square root fitting can identify non-ideal capillary flow behavior. For example, the microfluidic channel may have defects that can cause the cause the liquid sample to leak from a portion of the microfluidic channel. This can result in a deviation from the ideal capillary action; however, this deviation can be identified by the failure in square root fitting. Examples, of such non-ideal behavior are illustrated by the raw profiles at steep jumps shown in FIGS. 5A-11D. In one example, if such deviation occurred before or near the data collection time such as, but not limited to, 5 seconds to 30 seconds, such data was excluded in the final analysis.

In some embodiments, the method for detecting a perfluorooctanoic acid (PFOA) wherein using the microfluidic chip may include but is not limited to, collecting a set of data from the microfluidic chip via an image capture device, inputting the set of data into a computer programmable machine for analyzing the set of data. In some embodiments, the flow rate of a sample may be measured via the of images captured by an image capture device, transferred to a remote hard drive such as Google Drive, which are analyzed by a custom computer code, such as Python code. The custom computer code recognizes the channel layout, then recognizes the wetting front to evaluate the flow distances in pixels to generate a computer file containing the cumulative data of the flow distance over the time frame. In some embodiments, the computer file is a Google Sheets file containing the cumulative data of the flow distance over the time frame (30 frames per second).

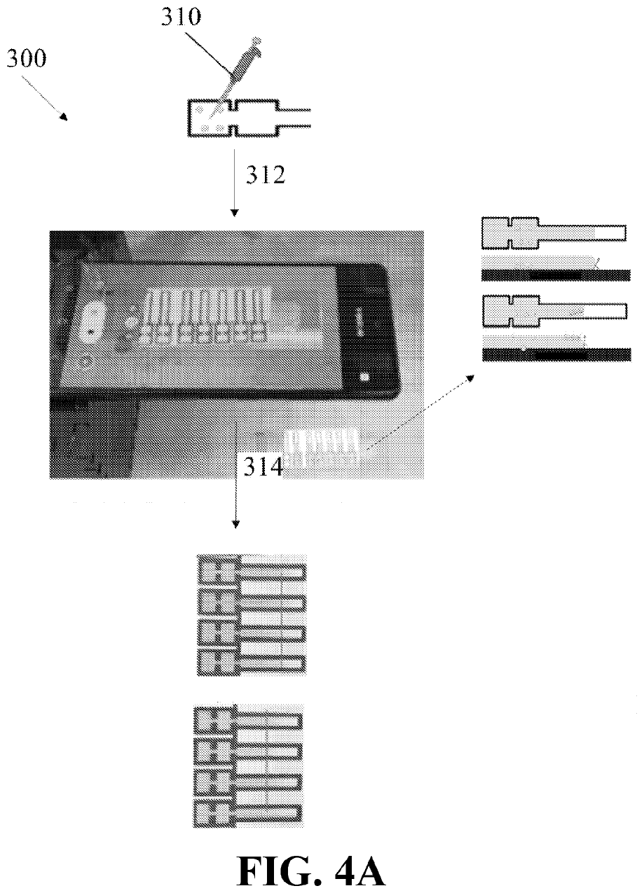

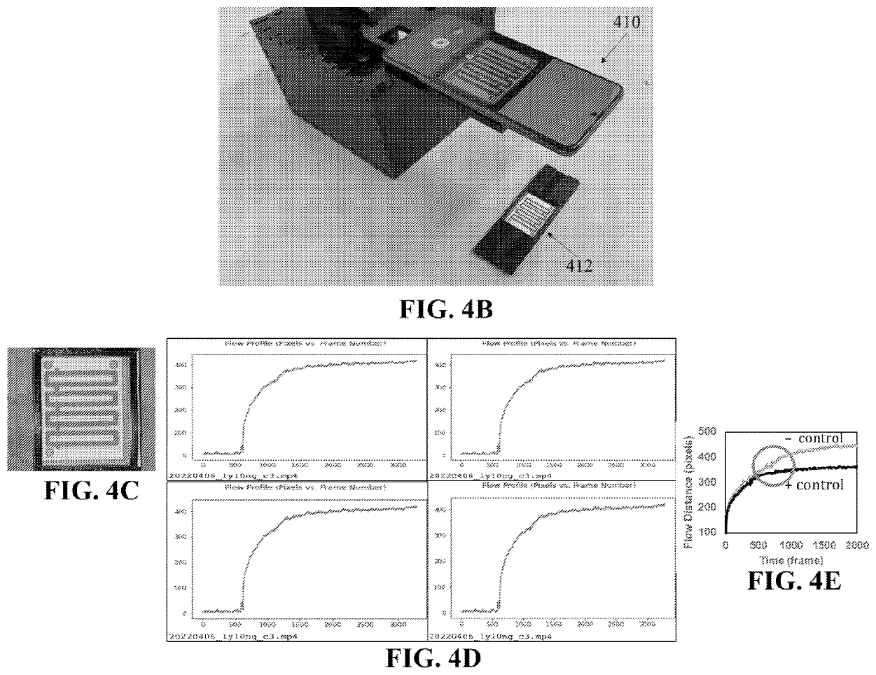

FIG. 4A is a flow diagram showing an aspect of the method for detecting one or more environmental toxicants in a liquid sample comprising: providing the liquid sample to the inlet of the paper microfluidic chip 310; measuring the flow distance of the liquid sample along the one or more microchannels 312; and analyzing a flow profile 314, wherein the flow profile comprises a plurality of measured flow distances against time.

FIGS. 4B-4E depicts an exemplary aspect of the method 400 disclosed herein for using the system also disclosed herein. With reference to FIG. 4B, a smartphone 410 was used to record a video of liquid flow through paper microfluidic chips 412. The paper microfluidic chip 412 comprising four channels depicted in FIG. 4C can be preloaded with a biomolecule such as, but not limited to, BSA, casein, or L-lysine. The video recording can be uploaded to remote could drive such as, but not limited to Google Drive from the smartphone and automatically analyzed using a custom computer code such as, but not limited to, Python. As shown by FIG. 4D a raw flow rate profile can be collected for the one or more microfluidic channels by obtaining flow distances from the one or more channels. Accordingly, the earliest time to make a statistically significant distinction between positive and negative controls is determined, for example, as illustrated in FIG. 4E, can vary from 5 seconds(s) to 30 s.

The present disclosure provides for a method of making a paper microfluidic chip for detecting environmental toxicant. In some aspects, the method for making a paper microfluidic chip for detecting an environmental toxicant comprises using a wax printer to print wax walls onto a cellulose paper, the wax walls defining at least one microfluidic channel on the cellulose paper; heating the cellulose paper at a temperature sufficient to melt the wax into the cellulose paper such that the wax walls extend through the cellulose from a first side to a second side of the cellulose paper, thereby forming a microchannel in the cellulose paper defined by the wax walls.

### VII. Overview of Several Aspects

Disclosed herein are aspects of a paper microfluidic chip, comprising cellulose fibers, at least one biomolecule, and a sample, wherein a sample interacts with the biomolecule to decrease the flow rate of the sample.

In any or all aspects, the sample comprises at least one perfluorinated-alkyl substance (PFAS).

In any or all aspects, the perfluorinated-alkyl substance is perfluorooctanoic acid (PFOA).

In any or all aspects, the sample interacts with the biomolecule via hydrophobic interactions, hydrogen binding, electrostatic repulsion, electrostatic attraction, or any combination thereof.

In any or all aspects, the at least one biomolecule is an amino acid, a polypeptide, a protein, or any combination thereof.

In any or all aspects, the biomolecule is L-lysine, bovine albumin serum, casein, or any combination thereof.

Also disclosed herein are aspects of paper microfluidic chip, comprising a paper substrate and at least one microchannel defined by wax walls, the paper substrate having a first side and a second side wherein the wax walls that define the at least one microchannel extend through the paper substrate from the first side to the second side thereby defining the microchannel in the paper substrate.

In any or all aspects, the paper microfluidic chip comprises a plurality of microchannels defined by wax walls.

In any or all aspects, the at least one microchannel having an inlet for receiving a sample.

In any or all aspects, the at least one microchannel further comprises at least one preloaded biomolecule located between the inlet and a location halfway along the length of the microchannel.

In any or all aspects, the preloaded biomolecule is an amino acid, a polypeptide, a protein, or any combination thereof.

In any or all aspects, the preloaded biomolecule L-lysine, bovine serum albumin, casein, or any combination thereof.

Also disclosed herein are aspects of a system comprising a paper microfluidic chip disclosed herein and image capture device.

In any or all aspects, the at least one microfluidic channel has at least one detection zone. In any or all aspects, the system further comprises a computer program for analyzing data collected by the image capture device.

In any or all aspects, the system further comprises a platform for securing the image capture device.

In any or all aspects, the system further comprises a paper microfluidic chip holder.

Also disclosed herein is a method comprising providing the paper microfluidic chip disclosed herein and using the paper microfluidic chip.

In any or all aspects, using the microfluidic chip comprises loading a sample that may contain a target molecule onto the microfluidic chip and measuring a flow rate of the sample along a microchannel in the microfluidic chip.

In any or all aspects, the method further comprises inputting the flow rate into a computer programmable machine and using the computer programmable machine to analyze the set of data to detect the presence or absence of the target molecule.

In any or all aspects, the target molecule is a PFAS.

In any or all aspects, the PFAS is Perfluorooctanoic acid.

Also disclosed herein is a method comprising using a wax printer to print wax walls onto a cellulose paper, the wax walls defining at least one microfluidic channel on the cellulose paper; and heating the cellulose paper at a temperature sufficient to melt the wax into the cellulose paper such that the wax walls extend through the cellulose paper from a first side to a second side of the cellulose paper, thereby forming a microchannel in the cellulose paper defined by the wax walls, further comprising loading a biomolecule into the microchannel, thereby forming a preloaded paper microfluidic chip for detecting environmental toxicants.

### VIII. Examples

Aspects of the present teachings can be further understood in light of the following examples.

Paper-Based microfluidic Chips: Using a standard wax printer (Xerox ColorCube 8580, Norwalk, CT, USA), the chip design was printed onto cellulose paper (Chromatography Paper 1; GE Healthcare, Chicago, IL, USA). The printed chips were placed onto a hot plate at 120° C. until the wax was melted through the paper depth, visibly showing opaque coloring on both sides of the chip. The chips were stored in a clean, sealed container before experimental use.

Reagent Dilutions: 0.1 mg of PFOA (Sigma-Aldrich, St. Louis, MO, USA) was dissolved into 10 mL of deionized (DI) water to make a stock solution of 10 ng/μL. Using serial dilutions into DI water, 100 pg/μL, 1 pg/μL, 100 fg/μL, 10 fg/μL, 1 fg/μL, 100 ag/μL, and 10 ag/μL PFOA samples were prepared. After every dilution, the solutions were vortexed for 10 seconds. The solutions of BSA, casein, L-lysine, Tween 20, SDS, and CTAB (all from Sigma-Aldrich) were prepared using the same protocol.

Assay Procedure: 3 μL of the reagent (BSA, casein, or L-lysine) solution (10 ng/μL or 0.001 ng/μL=1 pg/μL) was loaded onto the inlet (loading zone) of each channel on the chip. The solution flowed through each channel via capillary action. It was left dry for 20 min, when the channel appeared completely white again. The reagent-loaded paper chip was secured into the chip holder. The paper microfluidic chip had three square grids (used in QR codes), which were recognized by the code later. The paper microfluidic chip was placed into the 3D-printed chip holder (printed by Crealty Ender 3; Crealty 3D, Shenzhen, China) in line with the square grids. The smartphone was placed on a secure platform facing downwards. The smartphone's camera was set to video mode with the flash and grid view turned on. 3 μL of the sample (PFOA as a positive control, DI water as a blank, and non-fluorocarbon surfactants as negative controls) was loaded onto each chip channel's inlet (loading zone). Care was taken not to touch the pipette tip onto the channel. Loading was repeated for each channel on the chip. The video was recorded on the smartphone, for 2 min or until the liquid reached the end of each channel.

Data Processing: The videos were uploaded to Google Drive and analyzed using the FlowProfile_PFASModified.py code hosted in Google Colab. It is a custom Python code developed to automatically recognizes the channel layout and then the wetting front to evaluate the flow distances in pixels. Furthermore, it creates the Google Sheets file containing the cumulative data of the flow distance over the time frame (30 frames per second). The initial data before the flow started were removed from the Google Sheets labeled CleanedUp data.

Statistically significant differences between positive and negative controls could be observed as early as 5 s or as late as 30 s, depending on the reagent used. For example, The flow distances were collected at such time, e.g., 5 s, 10 s, 20 s, or 30 s, by taking the averages from 30 frames (1 s), e.g., 20 s to 21 s, which was one signal from a specific channel. Experiments were repeated multiple times using a different paper microfluidic channel, and the averages and standard errors were evaluated from those multiple experiments.

The entire flow profile, i.e., a cumulative data set of the flow distances (in pixels) against time (in frames), was also analyzed by fitting the data to a square root curve, following the Lucas-Washburn (L-W) equation for capillary flow. Square root curves were fitted in Origin (OriginLab; Northampton, MA, USA) and plotted in Microsoft Excel. The square root fitting was also useful for identifying non-ideal capillary flow behaviors. In some cases, the wax printing was not optimal, causing the liquid to “leak” from a certain point of each microfluidic channel. It leads to a deviation from the ideal capillary action, which can be identified by the failure in square root fitting. If such deviation occurred before or near the data collection time, e.g., 5 s to 30 s, such data were excluded in the final data analysis.

### Example 1

In this example three different reagents (BSA, casein, and L-lysine) at two different concentrations, 10 ng/μL 0.001 ng/μL (1 pg/μL), plus no reagent, i.e., seven different combinations, were tested for assaying three different PFOA concentrations plus a blank sample (DI water). Each combination was repeated three times. The flow distance (pixels) of different concentration of PFOA in DI water with different concentrations of preloaded reagents were compared. The time points used for each reagent varied depending on the flow rate profile.

After collecting the videos, a positive PFOA flow rate was plotted against a DI water flow rate to generate a flow rate profile (frame number (30 frames/s; 3000 frame=100 s) versus flow distance in pixels) with no preloaded reagent, 0.001 ng/μL L-lysine, 10 ng/μL L-lysine, 10 ng/μL casein, 0.001 ng/μL casein, 10 ng/μL BSA, BSA 0.001 ng/μL are shown in FIGS. 5A-11D. The flow rate profiles were compared when there was a first statistically significant difference between the positive and negative samples to determine a critical time (p<0.05).

FIG. 5A shows the raw flow rate profile of a sample comprising only DI water (NC) and no preloaded reagent; FIG. 5B shows the raw flow rate profile of a sample comprising 1 fg/μL PFOA with no preloaded reagent; FIG. 5C shows the raw flow rate profile of a sample comprising 10 fg/μL PFOA and no preloaded reagent, FIG. 5D shows the raw flow rate profile of a sample comprising 1000 fg/μL PFOA and no preloaded reagent. The flow distances were measured 60 seconds (nothing). The highest concentration (1000 fg/μL) of PFOA was statistically significantly different from the NC. However, the other two PFOA concentrations were not.

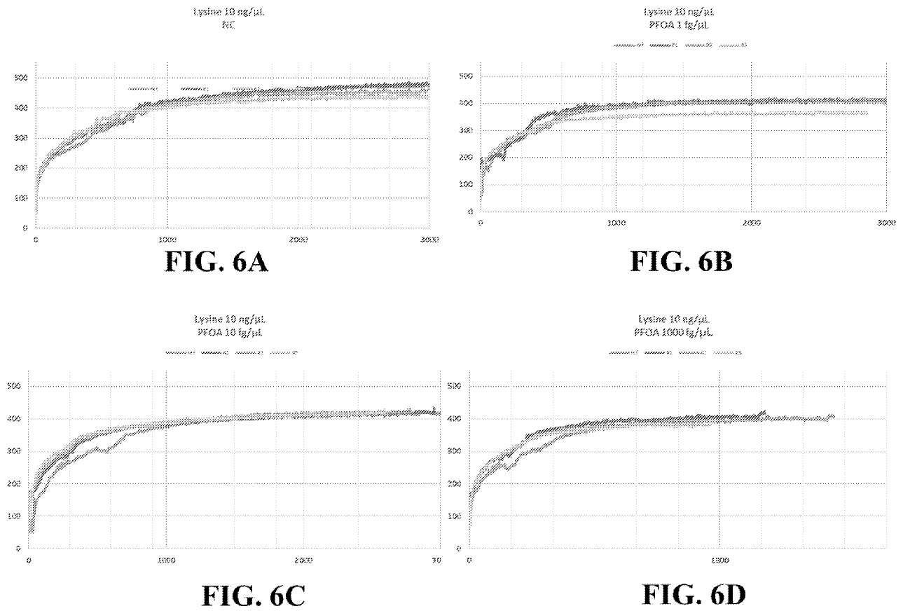

FIG. 6A shows the raw flow rate profile of a sample comprising only DI water (NC) and 10 ng/μL Lysine; FIG. 6B shows the raw flow rate profile of a sample comprising 1 fg/μL PFOA and 10 ng/μL Lysine; FIG. 6C shows the raw flow rate profile of a sample comprising 10 fg/μL PFOA and 10 ng/μL Lysine; FIG. 6D shows the raw flow rate profile of a sample comprising 1000 fg/μL PFOA and 10 ng/μL Lysine. Only 1000 fg/μL PFOA was statistically significantly different from the NC. Statistically significant differences were observed after 30 s, where the flow distances were measured, which were slightly shorter than “nothing.”

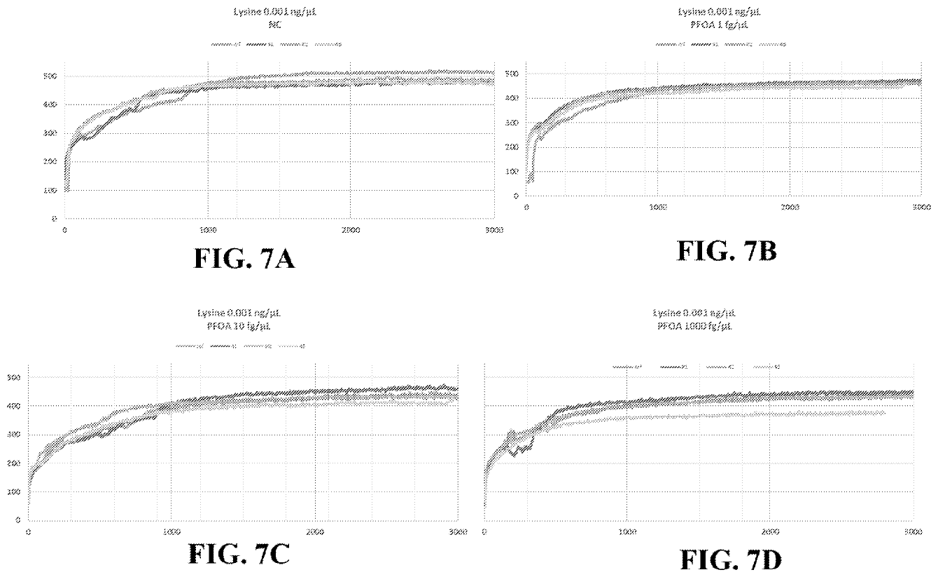

FIG. 7A shows the raw flow rate profile a sample comprising only DI water (NC) and 0.001 ng/μL Lysine, FIG. 7B shows the raw flow rate profile of a sample comprising 1000 fg/μL PFOA and 0.001 ng/μL Lysine, FIG. 7C shows the raw flow rate profile of a sample comprising 10 fg/μL PFOA and 0.001 ng/μL Lysine; FIG. 7D shows the raw flow rate profile of a sample comprising 1 fg/μL PFOA and 0.001 ng/μL Lysine. The flow distances were measured at 6 seconds for lysine 0.001 ng/μL. With 0.001 ng/μL L-lysine, 100 fg/μL PFOA additionally passed the t-test, potentially indicating L-lysine's ability to detect PFOA presence. In addition, significant differences were observed only after 6 s, where the flow distances were measured. This time was statistically significantly shorter than “nothing” and 10 ng/μL L-lysine, potentially indicating a desirable sensitivity.

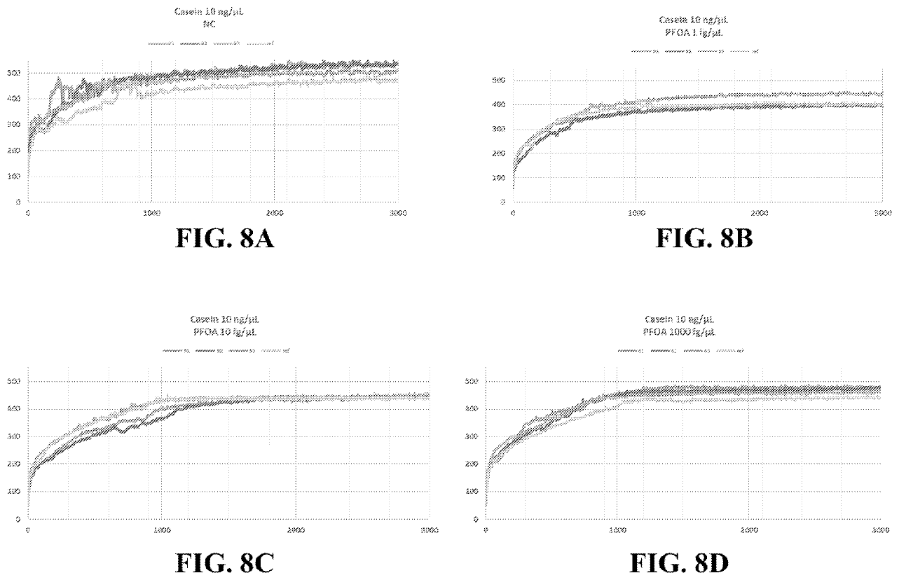

FIG. 8A shows the raw flow rate profile a sample comprising only DI water (NC) and 10 ng/μL casein; FIG. 8B shows the raw flow rate profile sample comprising 1 fg/μL PFOA and 10 ng/μL casein; FIG. 8C shows the raw flow rate profile sample comprising 1000 fg/μL PFOA and 10 ng/μL casein; FIG. 8D shows the raw flow rate profile sample comprising 1000 fg/μL PFOA and 10 ng/μL casein. Flow distances were measured at 30 seconds for 10 ng/μL casein. With 1 and 10 fg/μL PFOA was statistically significantly different from NC, but lost sensitivity at high PFOA concentration (1000 fg/μL). Significant differences were observed at 30 s, where the flow distances were measured.

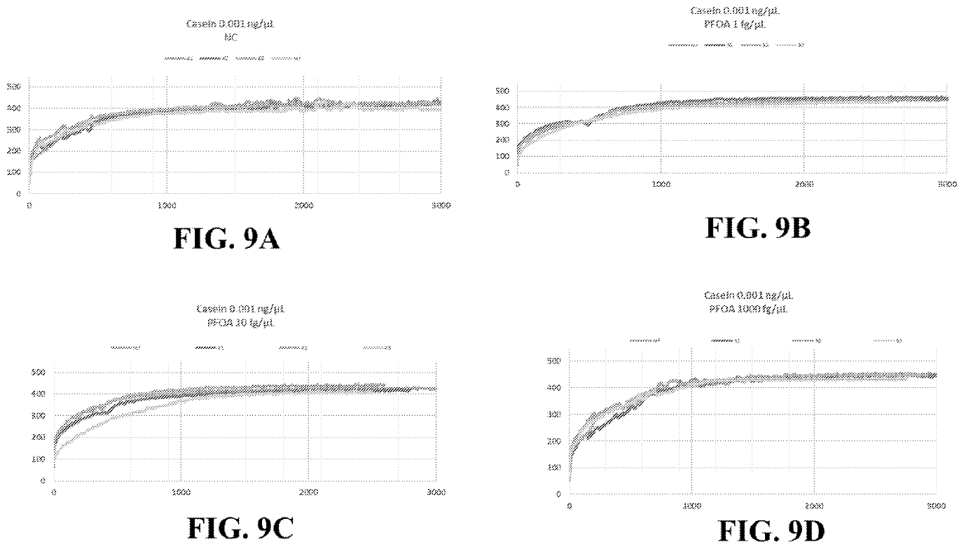

FIG. 9A shows the raw flow rate profile a sample comprising only DI water (NC) and 0.001 ng/μL casein; FIG. 9B shows the raw flow rate profile sample comprising 1 fg/μL PFOA and 0.001 ng/μL casein; FIG. 9C shows the raw flow rate profile sample comprising 10 fg/μL PFOA and 0.001 ng/μL casein, FIG. 9D shows the raw flow rate profile sample comprising 1000 fg/μL PFOA and 0.001 ng/μL casein. 0.001 ng/μL casein, on the other hand, did not show any significant difference from NC, although the flow distances could be measured as early as 5 s.

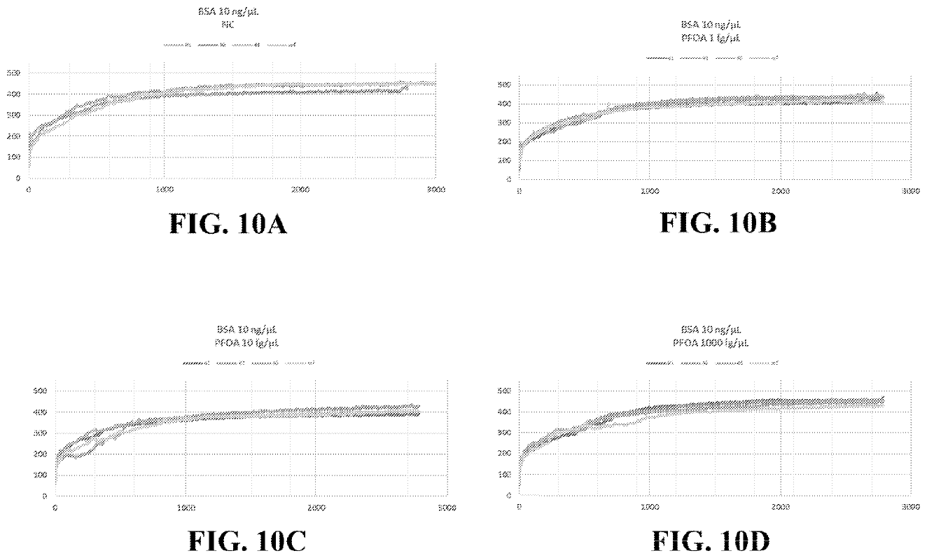

FIG. 10A shows the raw flow rate profile a sample comprising no PFOA and 10 ng/μL BSA; FIG. 10B shows the raw flow rate profile a sample comprising 1 fg/μL PFOA and 10 ng/μL BSA; FIG. 10C shows the raw flow rate profile sample comprising 10 fg/μL and 10 ng/μL BSA, FIG. 10D shows the raw flow rate profile sample comprising 1000 fg/μL PFOA and 10 ng/μL BSA. Statistically significant differences were determined at all PFOA concentrations. The flow distances could be measured at 20 s and 10 s, respectively. Therefore, 10 ng/μL BSA was chosen to investigate the assay range and the LOD.

FIG. 11A shows the raw flow rate profile sample comprising no PFOA and 0.001 BSA, FIG. 11B shows the raw flow rate profile a sample comprising 1 fg/μL and 0.001 BSA; FIG. 11C shows the raw flow rate profile sample comprising 10 fg/μL PFOA and 0.001 BSA, FIG. 11D shows the raw flow rate profile sample comprising 1000 fg/μL PFOA and 0.001 BSA. The flow distances were measured at 10 seconds for 0.001 ng/μL.

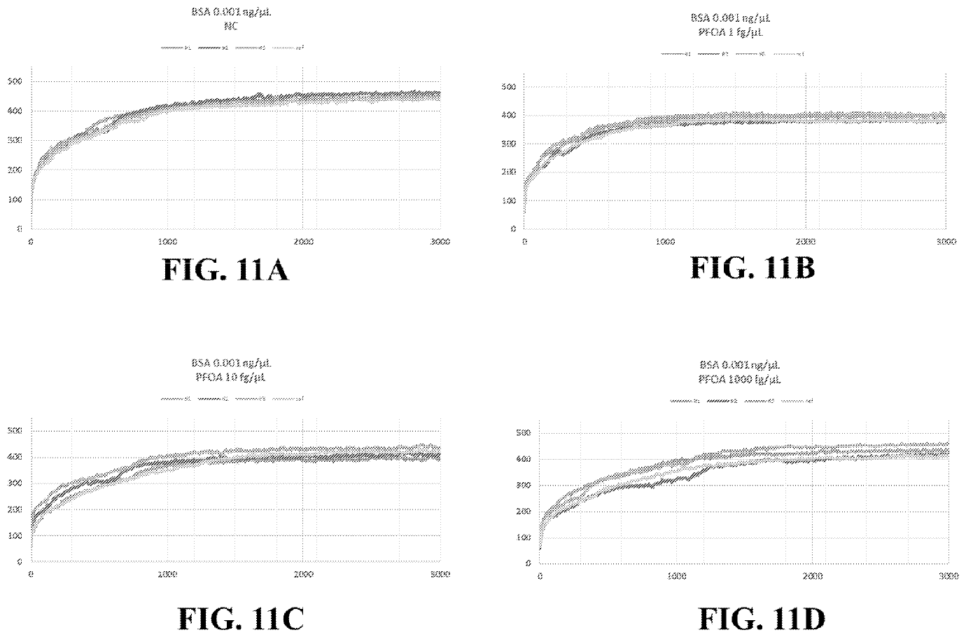

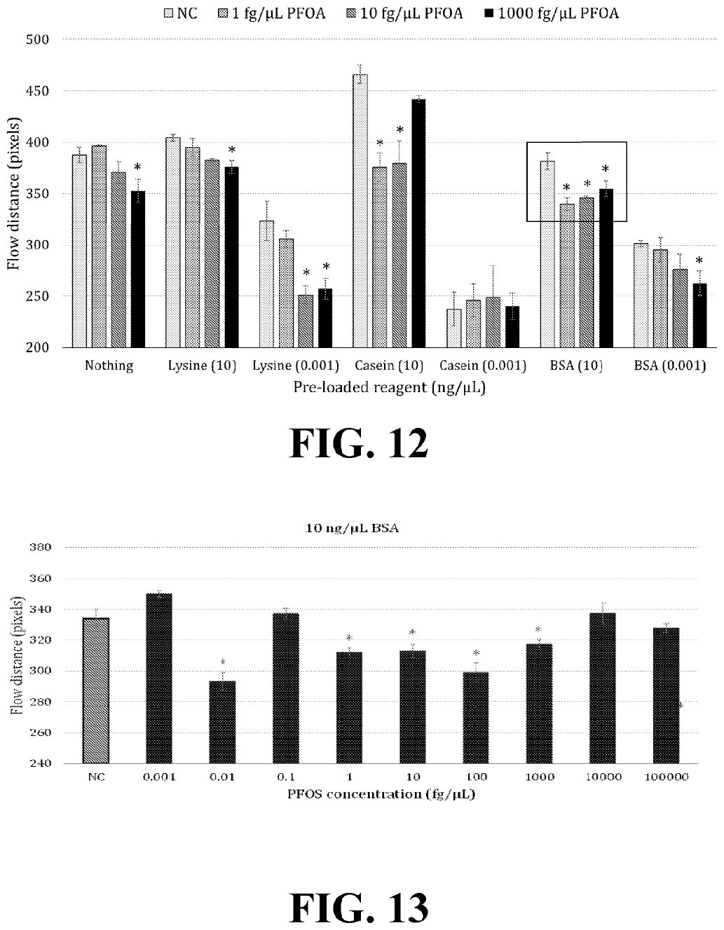

FIG. 12 is a bar graph showing the results of the samples comprising different concentration of PFOA in DI water of the flow distance (pixels) versus the different preloaded reagents (n=3, each time using a different paper microfluidic channel). Error bars represent standard errors and “*” indicates significant differences (p<0.05) from the NC in each data set. Noticeable differences from the NC (p<0.05) with 0.001 ng/μL L-lysine and 10 ng/μL BSA. The BSA results were more desirable as all three PFOA concentrations showed statistically significantly lower signals than the NC, while the L-lysine results showed significance only at 10 and 1000 fg/μL PFOA.

Similar results were obtained with 10 ng/μL lysine and 1000 fg/μL PFOA was statistically significantly different from the NC. Significant differences were observed after 30 seconds, where the flow distances were measured, which were slightly shorter than “nothing.” With 0.001 ng/μL lysine, 100 fg/μL PFOA additionally passed the t-test, potentially indicating L-lysine's ability to detect PFOA presence. In addition, differences were observed only after 6 seconds, where the flow distances were measured. This time was statistically significantly shorter than “nothing” and 10 ng/μL. L-lysine, potentially indicating a desirable sensitivity.

10 ng/μL casein showed improved results, with 1 and 10 fg/μL PFOA different from NC, but lost sensitivity at a PFOA concentration of 1000 fg/μL. Differences were observed at 30 s, where the flow distances were measured. In contrast 0.001 ng/μL casein did not show any significant difference from NC, although the flow distances could be measured as early as 5 s. 10 ng/μL BSA demonstrated differences at all PFOA concentrations whereas 0.001 ng/μL BSA did not. The flow distances could be measured at 20 s and 10 s, respectively. In view of this, 10 ng/μL BSA was chosen to investigate the assay range and the LOD.

### Example 2

In this example, nine different PFOA concentrations and a blank sample were again assayed (n=4-19 for each concentration) to determine the assay range and the LOD using a microfluidic chip preloaded with 10 ng/μL BSA. The concentration varied from 0.001 fg/μL (1 ag/μL) to 100 pg/μL at one order of magnitude increments.

The results of the assay are shown in FIG. 13 depicting a bar graph of the flow distance (pixels) versus the samples comprising PFOA concentrations of 0 (NC) 0.001 fg/μL, 0.01 fg/μL, 0.1 fg/μL, 1 fg/μL, 10 fg/μL, 100 fg/μL, 1000 fg/μL, 10,000 fg/μL, and 100,000 fg/μL. The Error bars represent standard errors. (*) indicates significant differences (p<0.05) from NC.

The lowest statistically significantly difference from NC was 0.01 fg/μL (10 ag/μL), which was the LOD. This LOD is below the recommended safety limit of 70 fg/μL. Above this LOD, no apparent linearity or correlation could be observed. Therefore, example demonstrated desirable qualitative results.

### Example 3

In this example, the assay of Example 2 was repeated with influent and effluent wastewater samples (n=12 each time using a different paper microfluidic channel), using 10 ng/μL BSA as the pre-loaded reagent. Influent wastewater samples are pre-processed sewage water samples that can contain food, fecal, pathogenic, and floral contaminants. Effluent samples are still classified as wastewater but have been filtered and cleaned.

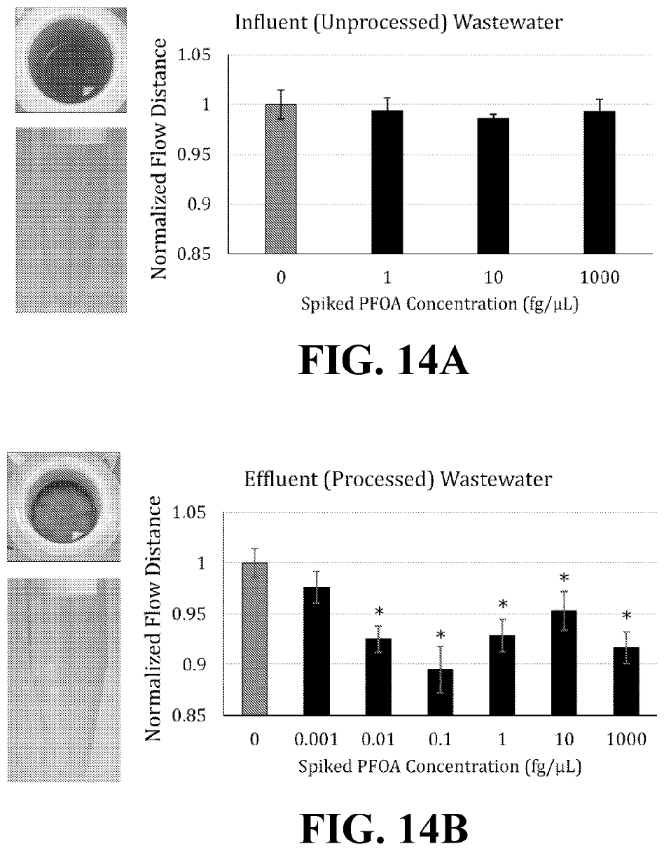

The Influent wastewater samples were spiked with PFOA concentrations of 1 fg/μL, 10 fg/μL, and 1000 fg/μL. Again, 10 ng/μL BSA was pre-loaded to each channel of the microfluidic chip and the flow distances were normalized to wastewater samples comprising no PFOA. FIG. 14A shows the assay results of PFOA spiked influent and shows top and side views of the wastewater sample tubes (10 mL) on the left (n=12). Error bars represent standard errors. (*) indicates statistically significant differences (p<0.05) from NC. With influent wastewater, there was a difference between 10 fg/μL PFOA and the unspiked influent wastewater. However, there was no significant statistical difference.

The effluent wastewater samples were spiked with PFOA concentrations of 0 fg/μL, 0.001 fg/μL, 0.01 fg/μL, 0.1 fg/μL, 1 fg/μL, 10 fg/μL, and 1000 fg/μL. With effluent wastewater, a desirable sensitivity was achieved down to 10 ag/μL PFOA, i.e., similar to the DI water results. FIG. 14B shows the results the assay results of PFOA for spiked effluent and shows top and side views of the wastewater sample tubes (10 mL) on the left (n=12). Error bars represent standard errors. (*) indicates statistically significant differences (p<0.05) from NC. In view of FIG. 14B, the spiked effluent wastewater had statistically significant differences for 0.01 fg/μL, 0.1 fg/μL, 1 fg/μL, 10 fg/μL, and 1000 fg/μL spiked PFOA samples. Therefore, the assay could be used with effluent wastewater.

### Example 4

Using the reagents from the optimization assays from Example 1, experiments with non-fluorocarbon surfactants, SDS, Tween 20, and CTAB at 10 fg/μL were repeated in this example. SDS was selected to represent an anionic surfactant with a strong negative charge. Tween 20 was also tested as a non-ionic (less toxic) surfactant, which still carries negative dipoles. All surfactants were assayed with varying the reagent type and concentrations (n=3 for each combination) and compared with the PFOA results.

The results are shown in FIG. 16 of the specificity assays with SDS, Tween 20, and CTAB, in comparison to PFOA (n=3, each time using a different paper microfluidic channel). 10 fg/μL data were used. Error bars represent standard errors. “*” indicates significant differences (p<0.05) from the NC in each data set.

With no pre-loaded reagent, no statistically significant differences were exhibited between the surfactants, PFOA, and the NC. With 10 ng/μL L-lysine, statistically significant differences from the NC were not observed for PFOA and the three surfactants. However, with 0.001 ng/μL L-lysine, statistically significant differences from the NC were observed for PFOA and CTAB but not for SDS and Tween 20. Additionally, 0.001 ng/μL L-lysine also worked to detect 10 fg/μL and 1000 fg/μL PFOA, as shown in FIG. 16. Therefore, 0.001 ng/μL L-lysine could be used to detect PFOA from SDS and Tween 20, but did not exhibit a desirable result for CTAB.

The results with 10 ng/μL casein were similar to 10 ng/μL L-lysine; both PFOA and surfactants were statistically significantly different from the NC and thus did not provide desirable specificity. The results with 0.001 ng/μL casein had no statistically significant differences between any of the combinations.

10 ng/μL BSA demonstrated statistically significant differences from the NC were observed for PFOA and SDS but not for Tween 20 and CTAB. Therefore, 10 ng/μL BSA could be used to detect PFOA from Tween 20 and CTAB, but not from SDS. The results with 0.001 ng/μL BSA did not show desirable sensitivity and specificity.

Therefore, this example demonstrated that 0.001 ng/μL L-lysine and 10 ng/μL BSA showed statistically significant differences between the negative control and PFOA and no differences with several non-fluorocarbon surfactants. However, 0.001 ng/μL L-lysine could not desirably distinguish PFOA from CTAB, and 10 ng/μL BSA could not desirably differentiate from SDS.

### Example 5

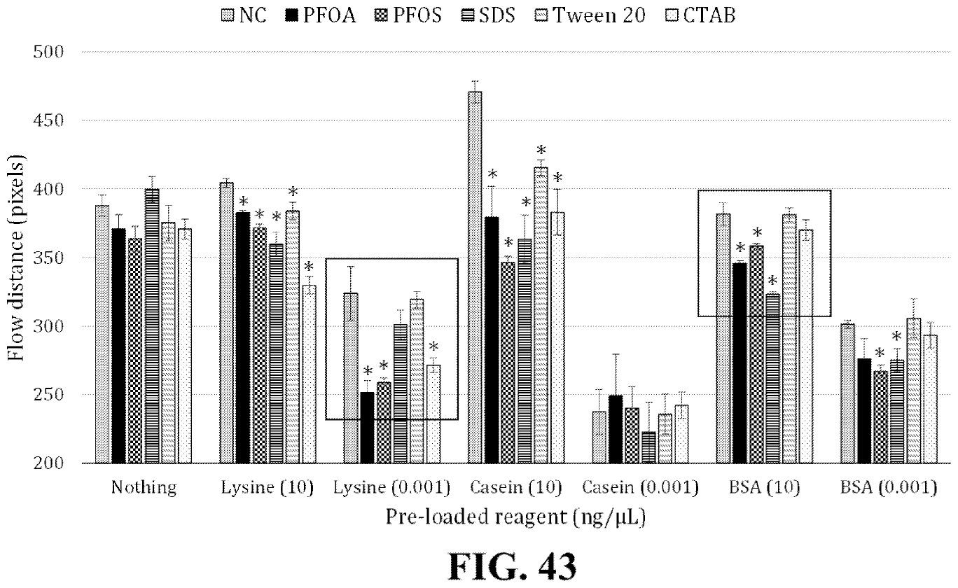

While desirable sensitive detection of PFOA was demonstrated with the LOD of 10 ag/Ml in DI water and effluent wastewater, the assay should demonstrate sufficient specificity over other non-fluorocarbon surfactants. In this example, the protocol shown in FIG. 43 was developed to assay NC, SDS, Tween 20, CTAB, and PFOA where “Pass” is defined as being statistically significantly different (p<0.05) from the NC in each data set. All surfactants were assayed with varying the reagent type and concentrations (n=3 for each combination) and compared with the PFOA results.

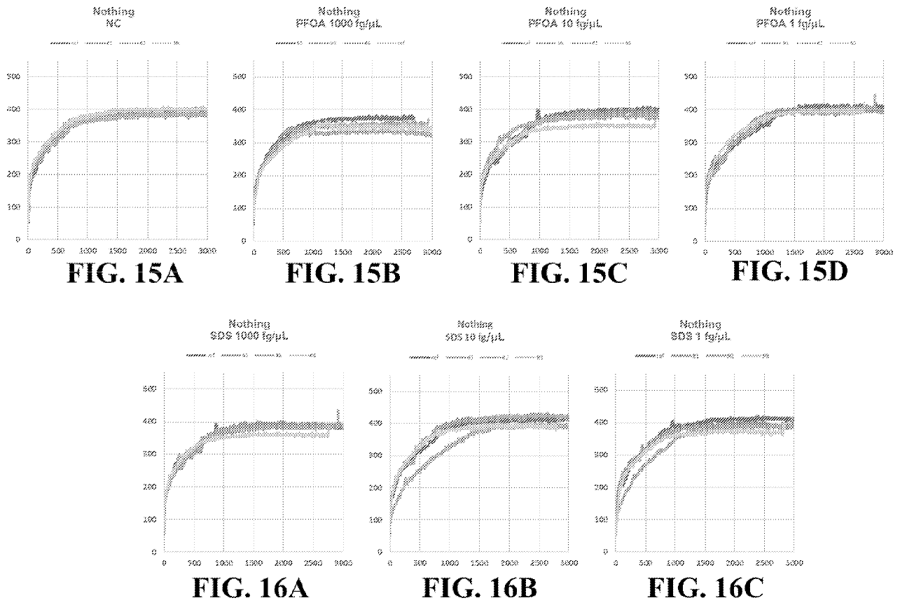

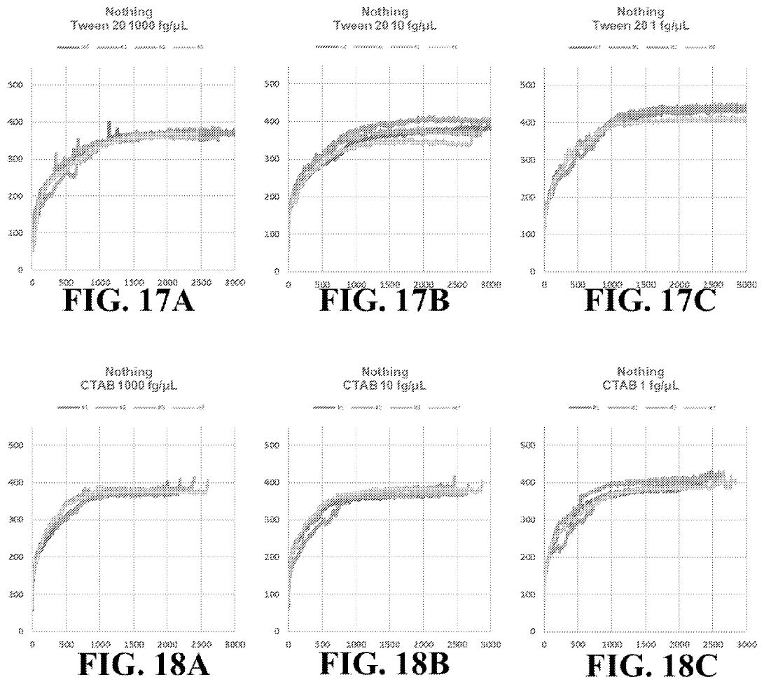

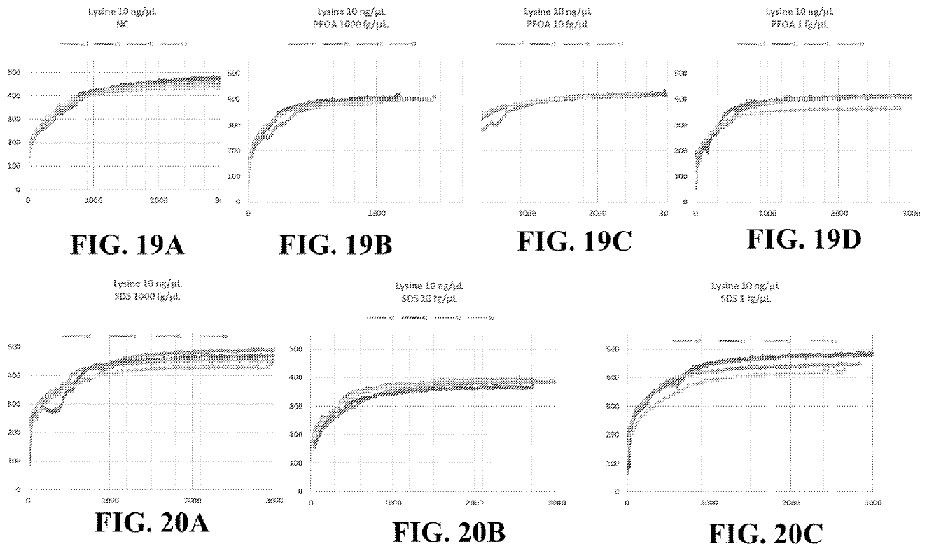

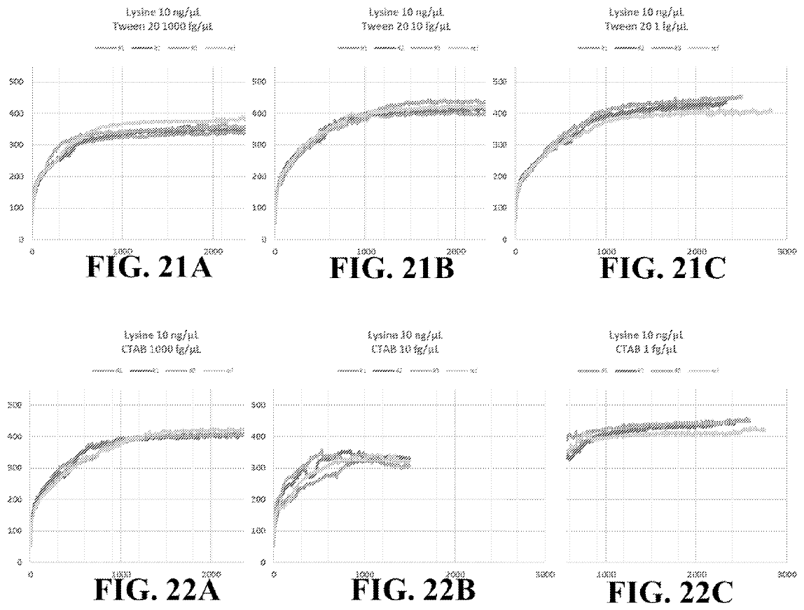

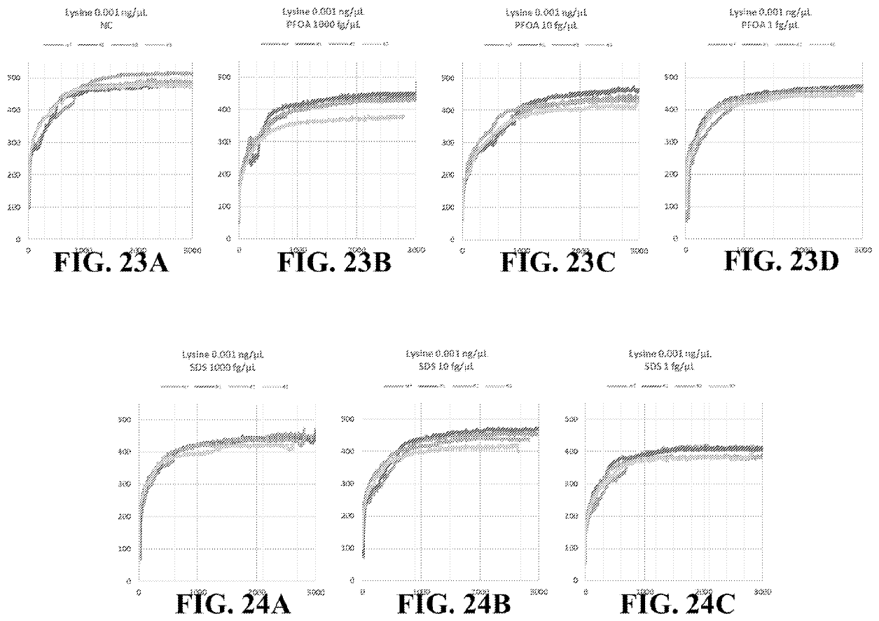

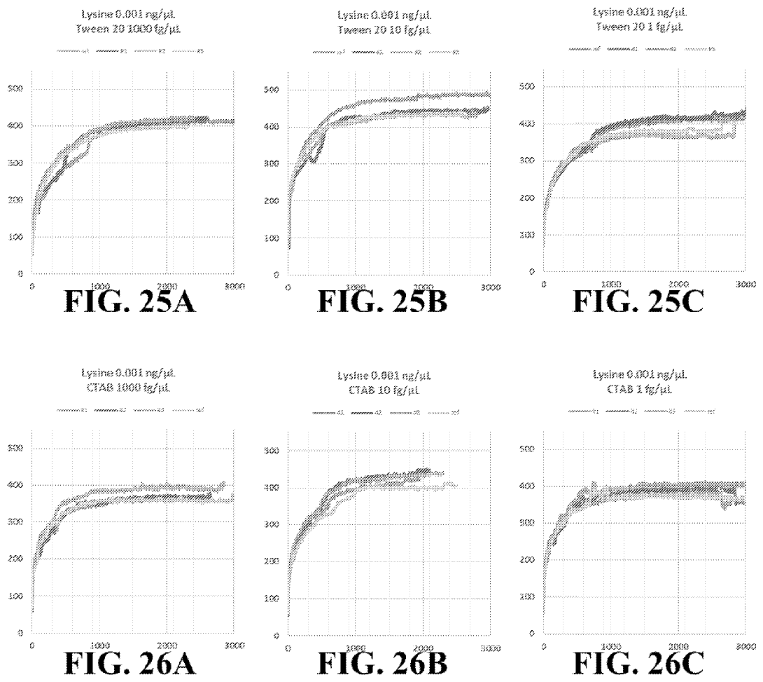

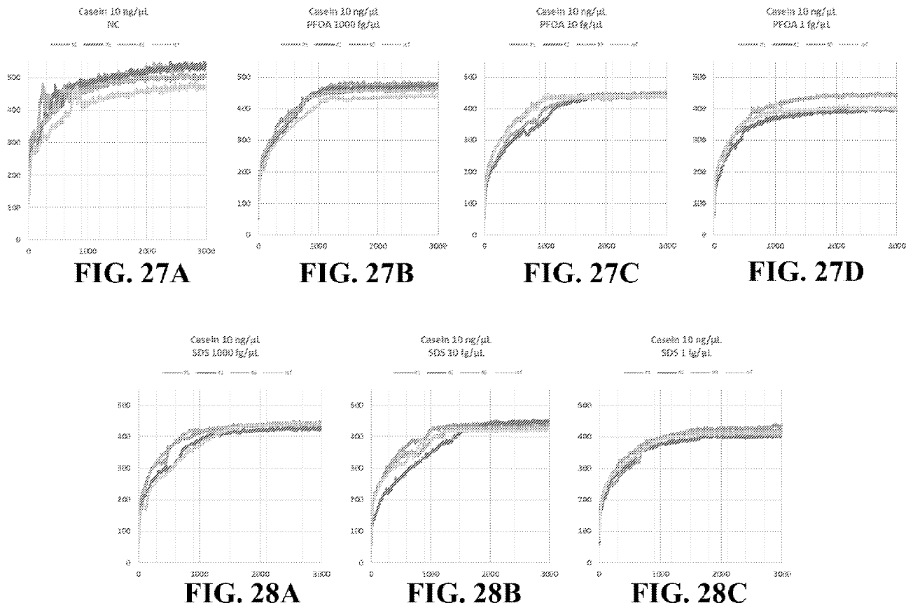

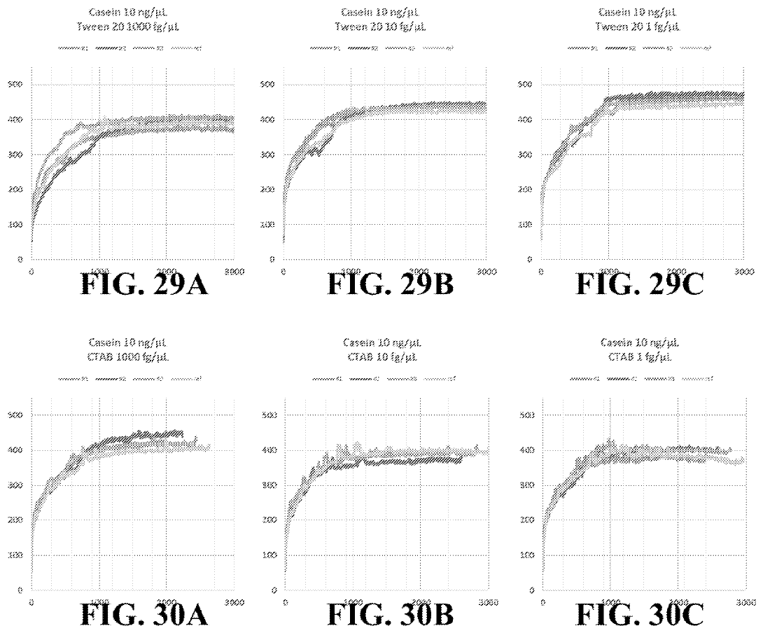

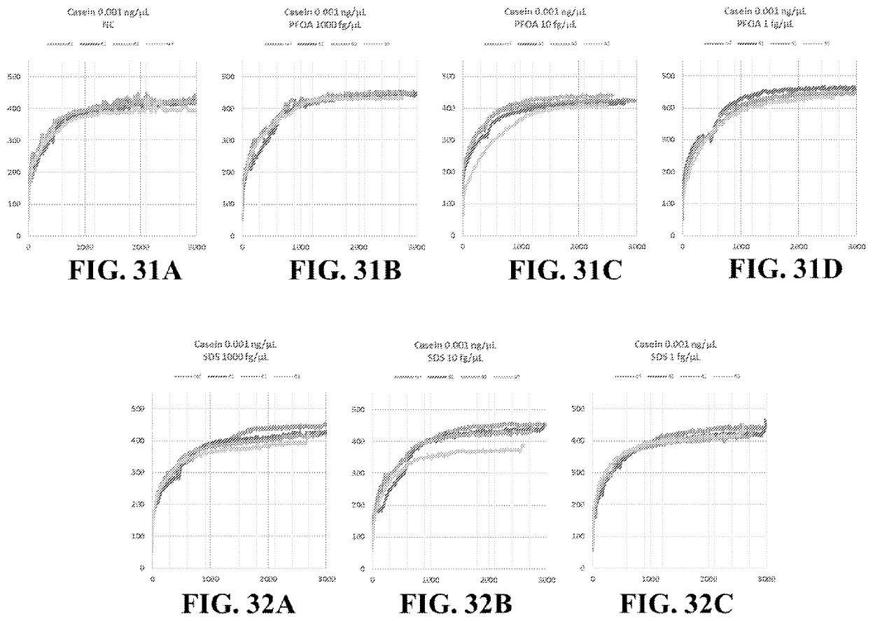

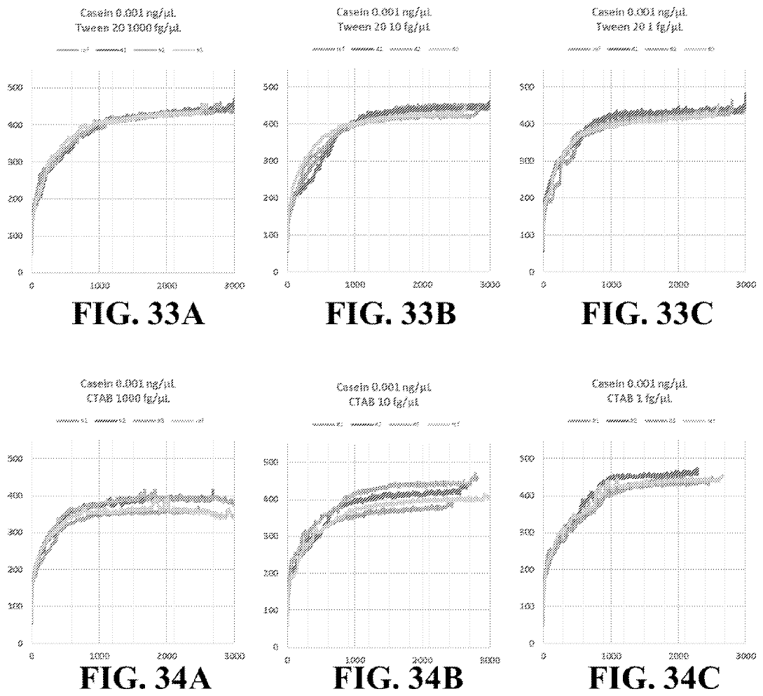

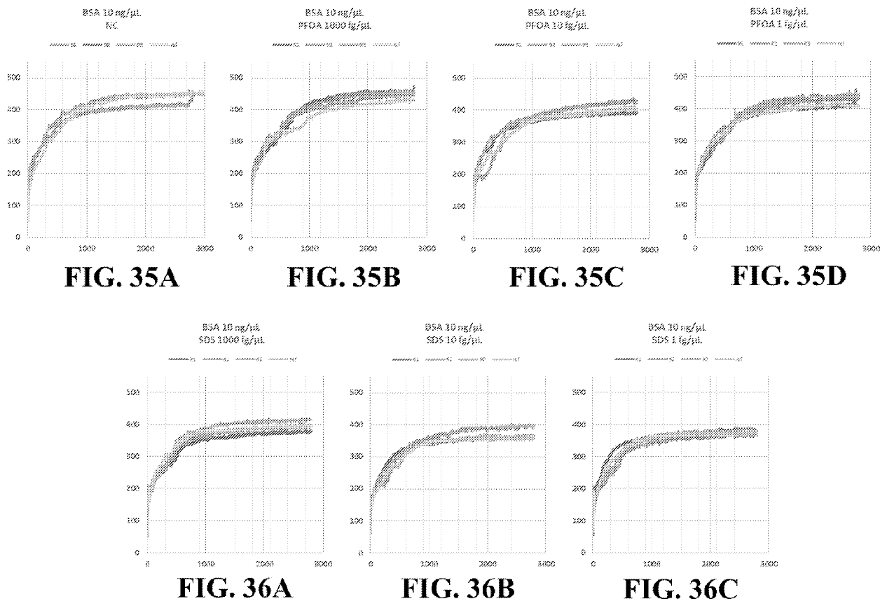

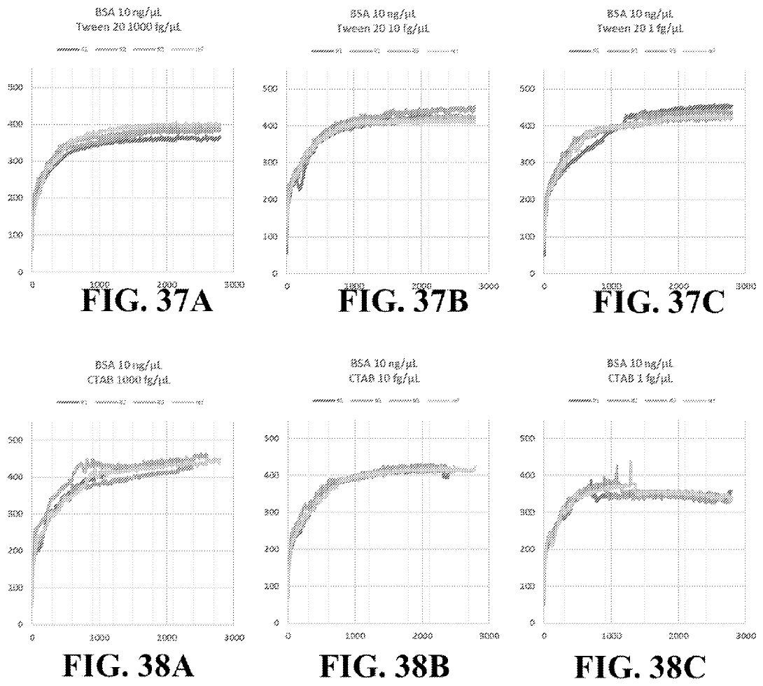

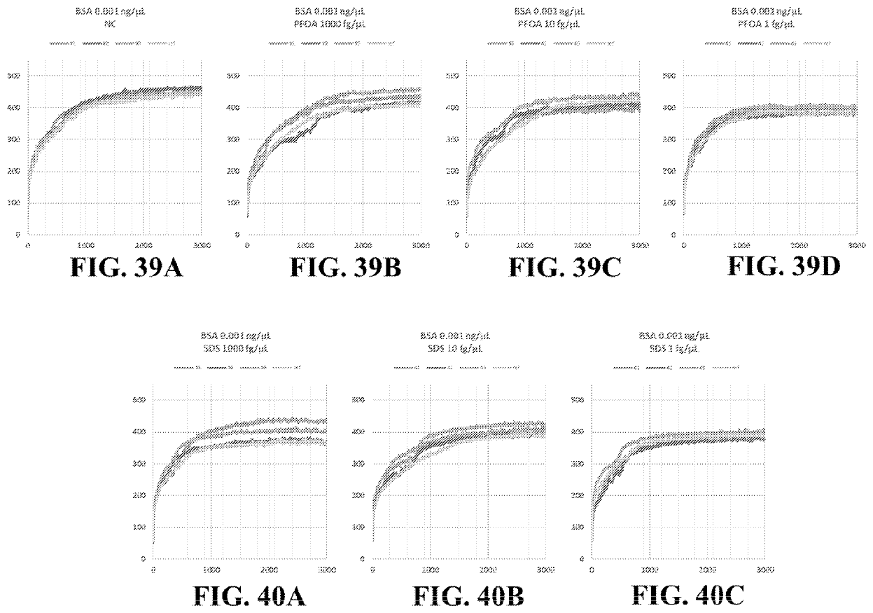

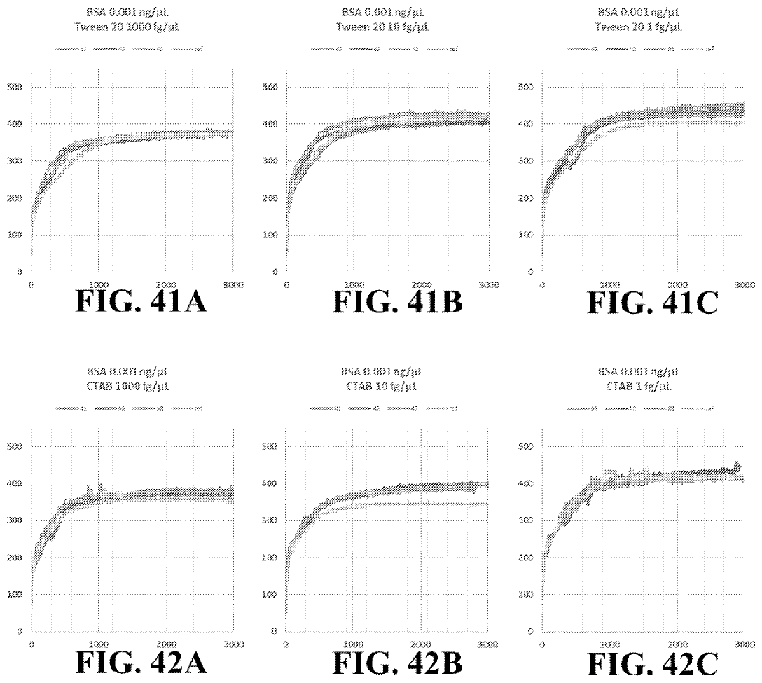

FIG. 43 shows the specificity assays with SDS, Tween 20, and CTAB, in comparison to PFOA (n=3, each time using a different paper microfluidic channel). 10 fg/μL data were used. Error bars represent standard errors and * indicates statistically significant differences (p<0.05) from the NC in each data set. FIGS. 15A-42C show the different flow rate profiles. FIG. 43 shows the specificity assays in comparison to PFOA.

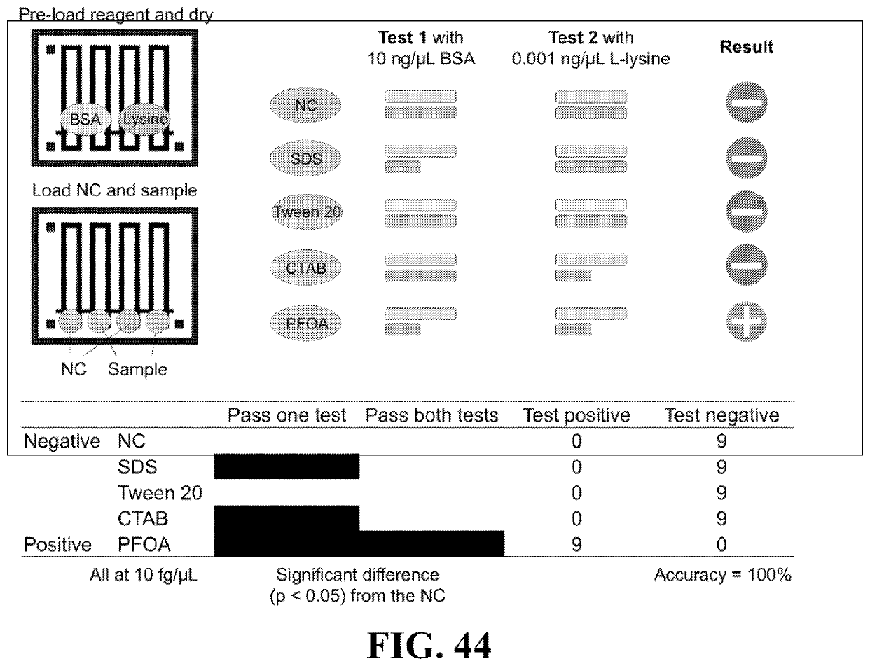

As shown in FIG. 44, all surfactants and NC were correctly classified as negatives and all PFOA samples as positives. Furthermore, the assay took less than 30 minutes to perform, including a 20-min reagent drying time. If the reagents are loaded and dried before the in-situ assays, the assay time can be reduced to 5 min, sample-to-answer. The samples flowed through the channel in less than 2 min. This assay does not require a target-specific antibody or MIP, as L-lysine and BSA are inexpensive from commercial vendors. Therefore, this example demonstrates unexpectedly rapid, economical, and efficient approaches in detecting the presence of PFOA.

In view of the many possible aspects to which the principles of the present disclosure may be applied, it should be recognized that the illustrated aspects are only preferred examples of the present disclosure and should not be taken as limiting the scope of the present disclosure. Rather, the scope of the present disclosure is defined by the following claims. We therefore claim as our present disclosure all that comes within the scope and spirit of these claims.

1. A composition for detecting one or more environmental toxicants in a liquid sample by decreasing a flow of the liquid sample comprising one or more environmental toxicants, comprising:
one or more cellulose fibers; and
one or more biomolecules associated with the one or more cellulose fibers, wherein the composition is configured so that when the composition contacts the liquid sample, the one or more biomolecules disassociate from the one or more cellulose fibers by interacting with the one or more environmental toxicants thereby decreasing the flow of the liquid sample comprising one or more environmental toxicants.

2. The composition of claim 1, wherein the one or more biomolecules comprise a first biomolecule, a second biomolecule that is different from the first biomolecule, and a third biomolecule that is different from the first biomolecule and the second biomolecule.

3. The composition of claim 1, wherein the one or more biomolecules is an amino acid, a polypeptide, a protein, or any combination thereof.

4. The one or more biomolecules of claim 3, wherein the one or more biomolecules is L-lysine, bovine serum albumin (BSA), casein, or any combination thereof.

5. The composition of claim 1, wherein the one or more cellulose fibers associate with the one or more biomolecules by mechanically capturing the biomolecule within the one or more cellulose fibers.

6. The composition of claim 1, wherein the one or more biomolecules interact with the one or more environmental toxicants by charge interaction, hydrophilic interactions, hydrophobic interactions, affinity interactions, hydrogen bonding, electrostatic repulsion, electrostatic attraction, Van der Waals forces, or any combination thereof.

7. The composition of claim 1, wherein the environmental toxicant is a perfluorinated-alkyl substance (PFAS).

8. The composition of claim 7, wherein the perfluorinated-alkyl substance is perfluorooctanoic acid (PFOA).

9. A composition, comprising:
one or more cellulose fibers;
a sample comprising one or more PFOAs; and
an interacting means for producing a PFOA conjugate upon interacting with the one or more PFOAs.

10. A paper microfluidic chip for detecting one or more environmental toxicants, comprising:
a paper substrate comprising one or more cellulosic fibers having a first side and a second side;
one or more biomolecules associated with the one or more cellulosic fibers; and
an inlet for receiving a liquid sample comprising one or more environmental toxicants, wherein the inlet is in communication with one or more microchannels defined by one or more walls, and wherein the walls that define the one or more microchannels extend through the paper substrate from the first side to the second side thereby defining one or more microchannels in the paper substrate.

11. The paper microfluidic chip of claim 10, wherein the one or more biomolecules is an amino acid, a polypeptide, a protein, or any combination thereof.

12. The paper microfluidic chip of claim 11, wherein the one or more biomolecules is L-Lysine, bovine serum albumin (BSA), casein, or any combination thereof.

13. A system, comprising:
the paper microfluidic chip according to claim 10;
an image capture device; and
a computer program for analyzing data collected by the image capture device.

14. The system of claim 13, wherein the image capture device collects data from the one or more microfluidic channels.

15. A method for detecting one or more environmental toxicants in a liquid sample, comprising:
providing the liquid sample to the inlet of the paper microfluidic chip according to claim 10;
measuring a flow distance of the liquid sample along the one or more microchannels; and
analyzing a flow profile, wherein the flow profile comprises a plurality of measured flow distances against time.

16. The method of claim 15, wherein the one or more environmental toxicant is PFOA.

17. The method of claim 16, wherein the PFOA concentration has a range greater than 0.05 fg/μL.

18. The method of claim 15, wherein analyzing the flow profile comprises fitting the data to a square root curve according to Equation 1:
$$L = \sqrt{\frac{tR {γ}_{LG} cos θ}{2 μ}} \qquad Equation 1$$
where: L is flow distance, t is time, R is capillary radius, γ_{LG} is surface tension at liquid-gas interface, θ is water contact angle, and μ is dynamic viscosity, and wherein R, γ_{LG}, θ, and μ are constant.

19. The method of claim 18, wherein an image capture device measures the flow distance in pixels and the time in frames, and wherein the flow profile is generated by fitting and plotting the square root curves via a computer programable machine.

20. A method for making the paper microfluidic chip of claim 10, comprising:
using a wax printer to print wax walls onto a cellulose paper, the wax walls defining at least one microfluidic channel on the cellulose paper;
heating the cellulose paper at a temperature sufficient to melt the wax into the cellulose paper such that the wax walls extend through the cellulose paper from a first side to a second side of the cellulose paper, thereby forming a microchannel in the cellulose paper defined by the wax walls; and
loading a biomolecule into the microchannel and thereby associate the one or more biomolecules with one or more cellulose fibers of the paper microfluidic chip.
